SEQUENCE LISTING (21 sequences)

SEQ ID NO: 1 - protein, 12 aa, Homo sapiens
ERKCCVECPPCP

SEQ ID NO: 2 - protein, 21 aa, Artificial Sequence
  Peptide antagonist of CXCR4
LGASWHRPDKCCLGYQKRPLP

SEQ ID NO: 3 - protein, 7 aa, Artificial Sequence
  Small peptide that binds FGF receptor- expressing cells
MQLPLAT

SEQ ID NO: 4 - protein, 8 aa, Artificial Sequence
  Octa peptide that blocks CD4 receptor binding by HIV
ASTTTNYT

SEQ ID NO: 5 - protein, 182 aa, Artificial Sequence
  G131 (2hinge-PD-1) PD-1 PD1L pathway interacting construct
METDTLLLWVLLLWVPGSTGERKCCVECPPCPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 6 - protein, 182 aa, Artificial Sequence
  G132 (PD-1-2hinge) PD-1 PD1L pathway interacting construct
METDTLLLWVLLLWVPGSTGPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVERKCCVECPPCP

SEQ ID NO: 7 - protein, 414 aa, Artificial Sequence
  G133 (2hinge-Fc-PD-1) G019-PD-1 PD-1 PD1L pathway interacting construct
METDTLLLWVLLLWVPGSTGERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 8 - protein, 399 aa, Artificial Sequence
  2hinge-CH2-CH3-PD-1 (G051-PD1) PD-1 PD1L pathway interacting construct
METDTLLLWVLLLWVPGSTGERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 9 - protein, 414 aa, Artificial Sequence
  PD-1-Fc-2hinge (PD-1- G045) PD-1 PD1L pathway interacting construct
METDTLLLWVLLLWVPGSTGPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 10 - protein, 414 aa, Artificial Sequence [sequence omitted]

SEQ ID NO: 11 - protein, 12 aa, Unknown
  CERVIG peptide for interaction with CD47/SIRPa pathway
CERVIGTGWVRC

SEQ ID NO: 12 - protein, 264 aa, Artificial Sequence
  G126 (Fc-CERVIG peptide) - G001-CERVIGTGWVRC (without multimerization domain) CD47/SIRPa pathway contruct
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKCERVIGTGWVRC

SEQ ID NO: 13 - protein, 276 aa, Artificial Sequence
  G122  (CERVIG peptide-Fc-2hinge) - CERVIGTGWVRC-G045 CD47/SIRPa pathway contruct
METDTLLLWVLLLWVPGSTGCERVIGTGWVRCEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 14 - protein, 276 aa, Artificial Sequence
  G123 ( 2hinge-Fc-“CERVIG” peptide)  - G019- CERVIGTGWVRC CD47/SIRPa pathway contruct
METDTLLLWVLLLWVPGSTGERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKCERVIGTGWVRC

SEQ ID NO: 15 - protein, 261 aa, Artificial Sequence
  G124 (2hinge-FcCH2-FcCH30“CERVIG”peptide) -G051 CERVIGTGWVRC CD47/SIRPa pathway contruct
METDTLLLWVLLLWVPGSTGERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKCERVIGTGWVRC

SEQ ID NO: 16 - protein, 44 aa, Artificial Sequence
  CERVIG-2h CD47/SIRPa pathway contruct
METDTLLLWVLLLWVPGSTGCERVIGTGWVRCERKCCVECPPCP

SEQ ID NO: 17 - protein, 69 aa, Artificial Sequence
  CERVIG-linker-2h CD47/SIRPa pathway contruct
METDTLLLWVLLLWVPGSTGCERVIGTGWVRCGGGGSGGGGSGGGGSGGGGSGGGGSERKCCVECPPCP

SEQ ID NO: 18 - protein, 257 aa, Artificial Sequence
  GB6500 (without multimerization domain) Her2/neu and FcGRIIIA interacting construct
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLYGDVSLTCLVKGFYPSDIAVEWESNGQPENNYKTTPPVLDSDGSFFLYSKLTVPRHSARMWRWAHGNVFSCSVMHEALHNHYTQKSLSLSPGK

SEQ ID NO: 19 - protein, 257 aa, Artificial Sequence; identical to SEQ ID NO: 18
  GB6545 (Modified Fc -2hinge) Her2/neu and FcGRIIIA interacting construct

SEQ ID NO: 20 - protein, 269 aa, Artificial Sequence
  GB6519 ( 2hinge - Modified Fc) Her2/neu and FcGRIIIA interacting construct
METDTLLLWVLLLWVPGSTGERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLYGDVSLTCLVKGFYPSDIAVEWESNGQPENNYKTTPPVLDSDGSFFLYSKLTVPRHSARMWRWAHGNVFSCSVMHEALHNHYTQKSLSLSPGK

SEQ ID NO: 21 - protein, 254 aa, Artificial Sequence
  GB6551 (2hinge-CH2-CH3 modified Fc) Her2/neu and FcGRIIIA interacting construct
METDTLLLWVLLLWVPGSTGERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLYGDVSLTCLVKGFYPSDIAVEWESNGQPENNYKTTPPVLDSDGSFFLYSKLTVPRHSARMWRWAHGNVFSCSVMHEALHNHYTQKSLSLSPGK

(19) United States
(12) Patent Application Publication
(10) Pub. No.: US 2014/0370012 A1
(43) Pub. Date: Dec. 18, 2014

(54) FUSION PROTEINS COMPRISING IGG2 HINGE DOMAINS

(71) Applicants: GLIKNIK INC., Baltimore, MD (US); University of Maryland, Baltimore, Baltimore, MD (US)

(72) Inventors: David Block, Baltimore, MD (US); Henrik Olsen, Baltimore, MD (US); Scott Strome, Reistertown, MD (US)

(73) Assignee: Gliknik Inc., Baltimore, MD (US)

(21) Appl. No.: 14/374,737

(22) PCT Filed: Jan. 28, 2013
(86) PCT No.: PCT/US2013/023404

§ 371 Date: Jul. 25, 2014

Related U.S. Application Data
(60) Provisional application No. 61/591,615, filed on Jan. 27, 2012.

(51) Int. Cl. C07K 16/28; C07K 14/74; A61K 39/395; A61K 45/06; C07K 14/705; C07K 14/46

(52) U.S. Cl. CPC C07K 16/2863; C07K 14/70521; C07K 14/70503; C07K 14/46; A61K 39/39533; A61K 45/06; C07K 14/70539; C07K 2319/30; C07K 2317/53; C07K 2317/41; C07K 2317/56; C07K 2317/55
USPC 424/134.1; 530/387.3

(57) ABSTRACT

The present invention relates to biologically active fusion proteins containing the IgG2 hinge as a multimerization domain capable of multimerizing proteins, peptides and small molecules which are active or more active in multimeric form; compositions comprising such fusion proteins; and methods of making and using such fusion proteins.

### CROSS REFERENCE TO RELATED APPLICATIONS

This application claims priority to U.S. Provisional Application No. 61/591,615, filed Jan. 27, 2012, the contents of which are herein incorporated by reference in their entirety.

### FIELD OF THE INVENTION

This invention relates generally to the fields of immunology, autoimmunity, inflammation, and tumor immunology. More specifically, the present invention relates to biologically active fusion proteins containing the IgG2 hinge as a multimerization domain capable of multimerizing proteins, peptides and small molecules which are active or more active in multimeric form; compositions comprising such fusion proteins; and methods of making and using such fusion proteins.

### SUMMARY OF THE INVENTION

There is a need for an alternative to currently existing methods for multimerizing therapeutic proteins and small molecules for use in immunotherapy that solves the problem of achieving the desired level of multimerization of the proteins, peptides, small molecules and carbohydrates/sugars while not causing an adverse reaction in the patient to a multimerization domain that is foreign to the patient's immune system. The present invention relates to biologically active fusion proteins comprising one or more naturally occurring multimerization domains and one or more peptides, proteins, small molecules, nucleic acids, fatty acids, or carbohydrates/sugars, compositions comprising the same and methods of using the same. These fusion proteins have broad application for treating a wide range of immunological, endocrinologic, inflammatory, infectious, and cancer disorders including, but not limited to autoimmune disease. Additionally, certain of these fusion proteins also have utility as laboratory reagents, such as for use in assays where biotin-streptavidin is currently used to make multimers, as an imaging agent, or as a clinical diagnostic agent.

In one embodiment, the present invention relates to a fusion protein comprising one or more IgG2 hinge domains of SEQ ID NO:1 and one or more peptides, proteins, small molecules, nucleic acids, fatty acids, or carbohydrates/sugars. In a further embodiment, the one or more IgG2 hinge domains multimerizes the one or more peptides, proteins, small molecules, nucleic acids, fatty acids, or carbohydrates/sugars into dimers or higher order multimers. In still a further embodiment, the one or more peptides, proteins or small molecules is any peptide, protein, small molecule, nucleic acid, fatty acid, or carbohydrate whose activity is improved by multimerization. In still a further embodiment, the observed improvement in activity is through increased affinity or avidity of binding of the multimerized compound to a protein. In one embodiment, the IgG2 domain of SEQ ID NO: 1 is fused to the C terminus of the one or more peptides or proteins. In another embodiment, the IgG2 domain of SEQ ID NO: 1 is fused to the N terminus of the one or more peptides or proteins. In another embodiment the IgG2 domain is fused to small molecules, nucleic acids, fatty acids, or carbohydrates/sugars. In yet another embodiment the IgG2 domain is fused to small molecules, nucleic acids, fatty acids, or carbohydrates/sugars through a linker protein such as the Fc domain.

In a specific embodiment, the fusion protein comprises one or more IgG2 hinge domains of SEQ ID NO: 1 and one or more proteins selected from cytokines, chemokines, hormones, cell surface receptors, cell surface receptor ligands, or monoclonal antibodies. In another embodiment the IgG2 hinge is fused to the extracellular domain of one or more proteins selected from cytokines, chemokines, hormones, cell surface receptors, or cell surface receptor ligands. In a particular embodiment, the IgG2 hinge fused to the extracellular domain of a cell surface receptor forms a soluble receptor. In one embodiment the soluble receptor is from the family of TNF receptors binding members of the TNF superfamily. In a particular embodiment, the one or more protein is one or more of PD-1, PD-1L, CTLA4, IL12, IL12RA, or major histocompatibility complex. In a particular embodiment, the IgG2 hinge fused to a hormone presents multimerized hormone to a hormone receptor. In one embodiment the multimerized hormone is insulin, human growth hormone, Glucagon-Like Peptide-1, leptin, orexin, ghrelin, or sex hormones. In another embodiment, the fusion protein comprises one or more IgG2 hinge domains of SEQ ID NO: 1 and one or more of the external domain of PD-1, CTLA4, the p40 subunit of IL12, or human parathyroid hormone. In another embodiment the IgG2 hinge is fused to synthetic peptides that bind to the extracellular domain of one or more proteins selected from cytokines, chemokines, hormones, cell surface receptors, cell surface receptor ligands.

In one embodiment, the fusion protein comprises one or more IgG2 hinge domains of SEQ ID NO: 1 and one or more PD-1 proteins. In one embodiment, the PD-1 protein comprises an extracellular domain of PD-1. In one embodiment the IgG2 hinge is fused to the C terminus of the PD-1 peptide. In another embodiment, the IgG2 hinge is fused to the N terminus of the PD-1 peptide. In still a further embodiment, the fusion protein also comprises an Fc domain in addition to the IgG2 hinge and PD-1 peptide. In still another embodiment, the fusion protein comprises a linker between the PD-1 peptide and the IgG2 hinge. In a further embodiment, the fusion protein is SEQ ID NO: 5, SEQ ID NO: 6, SEQ ID NO: 7, SEQ ID NO: 8, SEQ ID NO: 9, SEQ ID NO: 10.

In another embodiment, the fusion protein comprises one or more IgG2 hinge domains of SEQ ID NO: 1 and one or more peptides capable of binding SIRPα. In one embodiment, the peptide that binds SIRPα is a CD47 molecule. In yet another embodiment, the peptide that binds SIPRα is a CERVIG synthetic peptide. In a further embodiment, the CERVIG peptide that binds SIRPα comprises SEQ ID NO: 11. In one embodiment the IgG2 hinge is fused to the C terminus of the CERVIG peptide. In another embodiment, the IgG2 hinge is fused to the N terminus of the CERVIG peptide. In still a further embodiment, the fusion protein also comprises an Fc domain in addition to the IgG2 hinge and CERVIG peptide. In still another embodiment, the fusion protein comprises a linker between the CERVIG peptide and the IgG2 hinge. In a further embodiment, the fusion protein comprises SEQ ID NO: 13, SEQ ID NO: 14, SEQ ID NO: 15, SEQ ID NO: 16, SEQ ID NO: 17.

In yet another embodiment, the fusion protein comprises one or more IgG2 hinge domains of SEQ ID NO: 1 and one or more major histocompatibility complex proteins. In one embodiment, the one or more major histocompatibility complex proteins is a class I major histocompatibility complex. In another embodiment, the one or more major histocompatibility complex is a class II major histocompatibility complex. In still a further embodiment, the fusion protein comprising one or more major histocompatibility complex proteins further comprises a label. In one embodiment the label is a dye. In another embodiment, the label is a fluorophore. In one embodiment, the fusion protein comprising one or more IgG2 hinge domains and one or more major histocompatibility complex proteins is useful in tetramer assays, for example in detecting the presence of antigen specific T cells by flow cytometry.

In one embodiment, the fusion protein comprises one or more IgG2 hinge domains of SEQ ID NO: 1 and one ore more cell surface receptors. In one embodiment, the cell surface receptor is a G-protein coupled receptor. In a particular embodiment, the G protein coupled receptor is a chemokine receptor. In further embodiment, the chemokine receptor is CCR5, CXCR1, or CXCR2. In another embodiment, the cell surface receptor is a T cell receptor. In still another embodiment, the cell surface receptor is a B cell receptor. In still another embodiment, the cell surface receptor is a TNF super family receptor such as CD137, BAFF R, BCMA, CD27, CD30, CD40, DcR3, DcTRAIL, DR3, DR6, EDAR, Fas, GITR, HVEM, lyphotoxin beta R, NGF R, osteoprotegerin, OX40, RANK, RELT, TACI, TRAIL R, TROY, TWEAK R.

In another embodiment, the fusion protein comprises one or more IgG2 hinge domains of SEQ ID NO: 1 and one or more cell surface receptor ligands. In one embodiment, the cell surface receptor ligand is a TNF superfamily receptor. In a further embodiment the TNF superfamily receptor ligand is TNFα or BLyS. In a further embodiment, the cell surface receptor ligand is a ligand to a cell surface glycoprotein. In a further embodiment, the cell surface glycoprotein is a CD4, CD123, CD303, or a CD304 ligand.

In still a further embodiment, the fusion protein comprises one or more IgG2 hinge domains of SEQ ID NO: 1 and one or more of a chemotherapeutic agent, a cytotoxic molecule, a dye and/or a fluorophore. In yet a further embodiment, the fusion protein comprises one or more IgG2 hinge domains of SEQ ID NO: 1 and one or more of monosaccharides, disaccharides, oligosaccharides, polysaccharides, neoglycoproteins, glycoclusters, glycopolymers, monodisperse nanostructure termed glycodendrimers, sugar alcohols, and sugar-rods.

In another embodiment, the present invention relates to a fusion protein comprising one or more IgG2 hinge domains of SEQ ID NO: 1, one or more peptides, proteins, small molecules, nucleic acids, fatty acids, or carbohydrates/sugars and one or more immunoglobulin Fc domain monomers. In a further embodiment, the fusion protein contains one or more immunoglobulin Fc domains that is selected for low affinity binding to Fc gamma receptors. In a particular embodiment, the immunoglobulin Fc domain that is selected for low affinity binding to Fc gamma receptors is an IgG1, IgG2, IgG3 or IgG4 Fc domain. In a particular embodiment, the immunoglobulin Fc domain is mutated to bind poorly to Fc gamma receptors. In still a further embodiment, the Fc domain is mutated at positions 233, 234, 235, 236, 238, 239, 265, 269, 270, 292, 293, 295, 296, 297, 303, 327, 329, 338, 376, and/or 414, to bind poorly to Fc gamma receptors. In yet a further embodiment, the immunoglobulin Fc domain is modified, such as by one or more glycosylation changes relative to native human immunoglobulin Fc, to bind poorly to Fc gamma receptors. In a particular embodiment, the immunoglobulin Fc domain is modified by hyperfucosylation, demannosylation or hemi-glycosylation, thereby decreasing Fc receptor binding.

In another embodiment, the current invention relates to a fusion protein comprising one or more immunoglobulin Fc domains that have been engineered to comprise an antigen binding site and an IgG2 hinge that multimerizes the one or more Fc domains that have been engineered to comprise an antigen binding site. In a further embodiment, the IgG2 hinge is fused to the N terminus of the one or more Fc domains that have been engineered to comprise an antigen binding site. In another embodiment, the IgG2 hinge is fused to the C terminus of the one or more Fc domains that have been engineered to comprise an antigen binding site. In a further embodiment, the fusion protein further comprises a linker linking the IgG2 hinge with the Fc domain engineered to comprise an antigen binding site. In still a further embodiment the fusion protein comprises an addition Fc domain that has not been engineered to comprise an antigen binding site. In one embodiment, the one or more Fc domains that have been engineered to comprise an antigen binding site has been engineered to bind Her2/neu and comprises SEQ ID NO: 18. In a further embodiment, the fusion protein comprises SEQ ID NO: 19, SEQ ID NO: 20, SEQ ID NO: 21.

In another embodiment, the current invention relates to a fusion protein comprising one or more one or more antigen binding antibody variable domains, fragments or variants thereof and one or more IgG2 hinge monomers wherein the IgG2 hinge domains multimerize the one or more antigen binding antibody variable domains, fragments or variables thereof. In one embodiment, the one or more antigen binding antibody variable domains, fragments or variants thereof is a variable heavy chain linked to a variable light chain. In a further embodiment, the variable heavy chain linked to the variable light chain forms an epitope binding site. In a further embodiment, the variable heavy chain is linked to the variable light chain with a linker. In a further embodiment, the one or more one or more antigen binding antibody variable domains, fragments or variants thereof is a Fab fragment of a monovalent antibody, scFv, a diabody, a triabody, a minibody, a single-domain antibody, a nanobody or a single chain antibody. In a further embodiment, the IgG2 hinge is fused to the C terminus of the one or more one or more antigen binding antibody variable domains, fragments or variants thereof. In a further embodiment the IgG2 hinge is fused to the N terminus of the one or more one or more antigen binding antibody variable domains, fragments or variants thereof. In still a further embodiment, the fusion protein also comprises an Fc domain in addition to the IgG2 hinge and the one or more one or more antigen binding antibody variable domains, fragments or variants thereof. In still another embodiment, the fusion protein comprises a linker between the one or more one or more antigen binding antibody variable domains, fragments or variants thereof peptide and the IgG2 hinge.

In another embodiment, the current invention relates to a pharmaceutical formulation comprising a fusion protein containing one or more IgG2 hinge domains and one or more peptides, proteins, nucleic acids, fatty acids, carbohydrates or small molecules and optionally one or more immunoglobulin Fc domains and pharmaceutically acceptable excipients. In a further embodiment, the pharmaceutical formulation comprising the fusion protein is administered to a patient in need thereof intravenously, subcutaneously, orally, intraperitoneally, sublingually, ophthalmologically, buccally, intranasally, rectally, transdermally, by subdermal implant, or intramuscularly. In a further embodiment, the fusion protein is administered before, during or after administration with an additional pharmaceutically active agent. In a further embodiment the additional pharmaceutically active agent comprises a steroid; a biologic anti-autoimmune drug such as a monoclonal antibody, a fusion protein, or an anti-cytokine; a non-biologic anti-autoimmune drug; an immunosuppressant; an antibiotic; an antifungal; an anti-viral agent; a cytokine; or an agent otherwise capable of acting as an immune-modulator. In still a further embodiment, the steroid is prednisone, prednisolone, cortisone, dexamethasone, mometesone testosterone, estrogen, oxandrolone, fluticasone, budesonide, beclamethasone, albuterol, or levalbuterol. In still a further embodiment, the monoclonal antibody is infliximab, adalimumab, rituximab, tocilizumab, golimumab, ofatumumab, LY2127399, belimumab, veltuzumab, or certolizumab. In still a further embodiment, the fusion protein is etanercept or abatacept. In still a further embodiment, the anti-cytokine biologic is anakinra. In still a further embodiment, the anti-rheumatic non-biologic drug is cyclophosphamide, methotrexate, azathioprine, hydroxychloroquine, leflunomide, minocycline, organic gold compounds, fostamatinib, tofacitinib, etoricoxib, or sulfasalazine. In still a further embodiment, the immunosuppressant is cyclosporine A, tacrolimus, sirolimus, mycophenolate mofetil, everolimus, OKT3, antithymocyte globulin, basiliximab, daclizumumab, or alemtuzumab. In still a further embodiment, the fusion protein is administered before, during or after administration of a chemotherapeutic agent. In still a further embodiment, the fusion protein and the additional therapeutic agent display therapeutic synergy when administered together. In one embodiment, the fusion protein is administered prior to the administration of the additional therapeutic agent. In another embodiment, the fusion protein is administered at the same time as the administration of the additional therapeutic agent. In still another embodiment, the fusion protein is administered after the administration of the additional therapeutic agent.

In another embodiment, the current invention relates to a method of treating a patient in need thereof with an effective amount of a fusion protein containing one or more IgG2 hinge domains, one or more peptides, proteins, or small molecules and, optionally, one or more immunoglobulin Fc domains. In a further embodiment, the patient in need thereof has an inflammatory, infectious, neoplastic, hormonal, or autoimmune disease. In still a further embodiment, the inflammatory disease is selected from the group consisting of coronary artery disease, Alzheimer's Disease, Irritable Bowel Syndrome, and Non-alcoholic steatohepatitis. In still a further embodiment, the infectious disease is selected from among bacterial, viral, fungal, or prion infection. In a particular embodiment, the patient has sepsis. In yet a further embodiment, the autoimmune disease is selected from the group consisting of rheumatoid arthritis, multiple sclerosis, type I or type II diabetes mellitus, autoimmune thyroiditis, idiopathic thrombocytopenia purpura, autoimmune anemia, chronic inflammatory demyelinating polyneuropathy, multifocal motor neuropathy, scleroderma, systemic lupus erythematosus, psoriasis, inflammatory bowel disease including Crohn's Disease and Ulcerative Colitis, autoimmune uveitis, ANCA positive vasculitis, celiac disease, pemphigus, dermatopolymyositis, Goodpasture's Disease, Myasthenia gravis, Grave's Disease, Kawasaki Disease, sickle cell crisis, idiopathic pulmonary fibrosis, vitiligo, and atopic dermatitis. In yet a further embodiment, the autoimmune disease is associated with the transplantation of an organ from a donor to a recipient. In yet a further embodiment, the autoimmune disease is a disease that is not classically characterized as an autoimmune disease but in which cells of the immune system play an important role such as Alzheimer's disease, Parkinson's disease, Huntingdon's disease, osteopenia, and osteoporosis. In a particular embodiment, the hormonal disease is selected from diabetes, obesity, Addison's disease, Cushing's syndrome, acromegaly, polycystic ovary syndrome, hyperparathyroidism, hyperthyroidism, hypothyroidism, and osteoporosis.

In another embodiment, the fusion protein is administered to treat humans, non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds (e.g., chickens, turkeys, and ducks), fish and reptiles with species-specific or chimeric fusion proteins. In yet another embodiment, the human is an adult or a child. In still another embodiment, the fusion protein is administered to prevent autoimmune disease. In a further embodiment the fusion protein is administered to prevent vaccine-associated autoimmune conditions in companion animals and livestock.

### DETAILED DESCRIPTION OF THE INVENTION

The approach to multimerize peptides, proteins, small molecules, nucleic acids, and fatty acids that are active, or more active, as dimers and higher order multimers using the naturally occurring IgG2 hinge domain includes recombinant and/or biochemical creation of immunologically active fusion proteins which are surprisingly more efficient at multimerization than molecules multimerized using traditional multimerization techniques. The fusion proteins described herein have utility for treating, for example, autoimmune diseases, inflammatory diseases, endocrinologic diseases, cancer and infectious diseases including sepsis. Each embodiment is described in detail below along with specific exemplary embodiments.

As used herein, the use of the word “a” or “an” when used in conjunction with the term “comprising” in the claims and/or the specification may mean “one,” but it is also consistent with the meaning of “one or more,” “at least one,” and “one or more than one.”

As used herein, “fusion protein” refers to a contiguous polypeptide or molecule containing multiple domains fused or joined together to form a novel protein or protein small molecule/carbohydrate compound. For example, an exemplary “fusion protein” contains the IgG2 hinge domain of SEQ ID NO: 1, fused/joined to an immunoglobulin Fc domain or a modified immunoglobulin Fc domain, and further fused/joined to the external domain of CTLA4, to form the fusion protein. The term “fusion protein” also encompasses the fusion of an IgG2 hinge domain of SEQ ID NO:1 to a non-protein or peptide such as a small molecule or carbohydrate.

By “directly linked” is meant two sequences connected to each other without intervening or extraneous sequences, for example, restriction enzyme recognition sites or cloning fragments. One of ordinary skill in the art will understand that “directly linked” encompasses the addition or removal of amino acids so long as the multimerization capacity is substantially unaffected. In certain embodiments, the IgG2 hinge is directly linked to a peptide, protein, nucleic acid, fatty acid, or small molecule. In other embodiments, the IgG2 hinge is directly linked to an immunoglobulin Fc domain monomer which is in turn directly linked to a peptide, protein, nucleic acid, fatty acid, or small molecule. In other embodiments, the peptide, protein, nucleic acid, fatty acid, or small molecule that is directly linked to the IgG2 hinge is in turn directly linked to an immunoglobulin Fc domain monomer.

By “homologous” is meant identity over the entire sequence of a given nucleic acid or amino acid sequence. For example, by “80% homologous” is meant that a given sequence shares about 80% identity with the claimed sequence and can include insertions, deletions, substitutions, and frame shifts. One of ordinary skill in the art will understand that sequence alignments can be done to take into account insertions and deletions to determine identity over the entire length of a sequence.

The following paragraphs define the building blocks of the fusion proteins of the present invention, both structurally and functionally, and then define the fusion proteins themselves. However, it is first helpful to note that, as indicated above, each of the fusion proteins of the present invention has at least one IgG2 hinge domain. The IgG2 hinge domain interacts with an IgG2 hinge domain on another fusion protein to create multimers. Therefore, the most functional form of the fusion proteins and discussed herein generally exist in a dimeric (or multimeric) form. The monomers of the fusion proteins discussed herein are the single chains that must associate with at least a second chain to form a functional homodimeric structure and multimeric structure.

### IgG2 Hinge

As is known in the art, the human IgG2 can form covalent dimers through the hinge region (Yoo, E. M. et al. J. Immunol. 170, 3134-3138 (2003); Salfeld Nature Biotech. 25, 1369-1372 (2007)). The dimer formation of IgG2 is potentially mediated through the IgG2 hinge structure by C-C bonds (Yoo et al 2003), suggesting that the hinge structure alone can mediate dimer formation. The amount of IgG2 dimers found in human serum, however, is limited. It can be estimated from an SDS-PAGE gel that the amount of IgG2 existing as a dimer of the homodimer is less than 10% of the total IgG2 (Yoo et al. 2003). Furthermore, there is no quantitative evidence of the multimerization domain of IgG2 beyond the dimer of the homodimer. (Yoo et al. 2003). That is, native IgG2 has not been found to form higher order multimers in human serum. Therefore, the results presented herein are surprising in the degree of multimerization relative to native IgG2 and particularly surprising in that the IgG2 hinge-containing fusion proteins may be present in high order multimers. The amino acid sequence of the human IgG2 hinge monomer is as follows: ERKCCVECPPCP (SEQ ID NO: 1). We have demonstrated that mutation of any one of the 4 cysteines in SEQ ID NO: 1 may be associated with greatly diminished multimerization of the fusion protein. There are two C-X-X-C portions of the IgG2 hinge monomer referred to herein as “amino acid cores.” Thus, fusion protein monomers of the present invention may comprise either the complete 12 amino acid sequence of the IgG2 hinge monomer, or either or both of the four amino acid cores along with Fc domain monomers. While the X-X of the amino acid core structures can be any amino acid, in a preferred embodiment the X-X sequence is V-E or P-P. The skilled artisan will understand that the IgG2 hinge monomer may be comprised of any portion of the hinge sequence in addition to the core four amino acid structure, including all of the IgG2 hinge sequence. Thus, the IgG2 hinge sequence may comprise all 12 amino acids of SEQ ID NO: 1, or any combination of amino acids while maintaining the two C-X-X-C motifs, so long as the IgG2 hinge maintains its ability to multimerize the protein, peptide, nucleic acid, fatty acid, or small molecule. For example, the IgG2 hinge may comprise C-X-X-C-X-X-C alone or with any combination of amino acids flanking the core structure. Without being bound by theory, the IgG2 hinge of one fusion protein may bind the IgG2 hinge of another fusion protein, thereby forming a dimer of the homodimer, or higher order multimers while retaining increased activity such as functional binding to receptors compared to the unmultimerized proteins. Alternatively, the C-X-X-C motifs in the IgG2 hinge may form C-C bridges with C's in other protein domains thereby forming a dimer of the homodimer, or higher order multimers while retaining increased functional binding to receptors compared to the unmultimerized proteins. Without being bound by theory, the multimers formed through C-C bridges are stable and comprise covalent bonds on disulfide bonding analysis.

### Peptides, Proteins, Nucleic Acids, Fatty Acids, and Small Molecules

The peptides, proteins, nucleic acids, fatty acids, and small molecules useful in the present invention are those that show improved or increased activity when multimerized. Examples of proteins whose activity is improved by multimerization include, for example, monoclonal antibodies, bispecific antibodies, members of the TNFR superfamily (such as 4-1BB, APRIL, BAFF, TRAIL, BLyS, LIGHT, Lymphotoxin, Lymphotoxin beta, TRANCE, TWEAK, TNF-alpha, TNF-beta, CD27 ligand, CD30 ligand, CD40 ligand, EDA, EDA-A1, EDA-A2, FAS ligand, GITR ligand, OX40 ligand, and TLA), an interferon (such as IFNA1 (Interferon α1), IFNA2, IFNA4, IFNA5, IFNA8, IFNB1, IFNG (Interferon γ), and IFNK), an interleukin (such as IL10, IL11, IL12A, IL12B, IL13, TXLNA, IL15, IL16, IL17A, IL17B, IL17C, IL25 (IL17E), IL8, IL9, IL1A, IL1B, IL1F10, IL36RN, IL36A, IL37, IL36B, IL36G, IL2, IL20, IL21, IL22, IL24, IL3, IL4, IL5, IL6, IL7, IL8, IL9), a chemokine (such as MCP-1, MIP-1a, MIP-1b, RANTES, cotaxin, MPF-1, CXCL-17, CXCL-10, CXC3) a bone morphogenic proteins and TGF-β family member (such as BMP1, BMP2, BMP3, BMP4, BMP5, BMP6, BMP7, BMP8B, GDF10 (BMP3B), GDF11 (BMP11), GDF2 (BMP9), GDF3, GDF5, MSTN, GDF9, INHA, INHBA, NODAL, TGFA, TGFB1, TGFB2, and TGFB3), any PDGF/VEGF Family (such as FIGF (VEGFD) and PDGFA), other cytokines (such as CSF1, (MCSF), FAM3B, LEFTY2), a soluble receptor (such as any of the 109 soluble receptors listed in the R&D Systems Hematopoietic Array and Common Analytes Array), a cell surface receptor including any Cluster of Differentiation, or a G-protein coupled receptor such as chemokine receptors (such as CCR5, CXCR1, and CXR2), TNF Super family receptors (such as CD137, BAFF R, BCMA, CD27, CD30, CD40, DcR3, DcTRAIL, DR3, DR6, EDAR, Fas, GITR, HVEM, lyphotoxin beta R, NGF R, osteoprotegerin, OX40, RANK, RELT, TACI, TRAIL R1, TRAIL R2, TRAIL R3, TRAIL R4, TROY, or TWEAK R), ligands to a cell surface receptor including any cluster of differentiation (CD4, CD303 or CD304), a naturally occurring hormone (such as α-MSH, GLP-1, insulin, human growth hormone, glucagon, insulin-like growth factor-1, leptin, erythropoietin, thyroid stimulating hormone, follicle stimulating hormone, prolactin, leutinizing hormone, vasopressin, oxytocin, adrenocorticotropic hormone, thyrotropin releasing hormone, gonadotropin releasing hormone, growth hormone releasing hormone, corticotropin releasing hormone, somatostatin, melatonin thyroxine, calcitonin, parathyroid hormone, phosphatonin, osteocalcin, glucocorticoids such as cortisol, mineralocorticoids such as aldosterone, androgens such as testosterone and DHEA, estrogens such as estradiol, progestins such as progesterone, amylin, human chorionic gonadotropin, calcitriol, calciferol, gastrin, secretin, atrial naturetic peptide, cholecystokinin, incretins, fibroblast growth factor 19, neuropeptide Y, ghrelin, PYY 3-36, angiotensinogen, thrombopoeitin, hepcidin, retinol binding protein 4, and adiponectin), neurotransmitters (such as epinephrine, norepinephrine, serotonin, acetylcholine, glutamate, glycine, aspartate, GABA, nitric oxide, histamine, dopamine, trace amines that bind to TAAR receptors, GHB that binds to GABAb receptor, hypocretin, niacin, endocannabinoids such as anandamine, 2-AG, noladin ether, NADA, and OAD, and endogenous opioids such as enkephalin, beta-endorphin, dynorphin, endomorphin, nociceptin, opiorphin, and morphine), growth factors (such as granulocyte macrophage colony stimulating factor, epidermal growth factor, fibroblast growth factor, and platelet derived growth factor), a fragment of any thereof, a functional analogue of any thereof, a functional analogue of a fragment of any thereof, and any combination thereof. Examples of peptides whose activity is improved by multimerization include, for example, the external domain of CTLA4, the p40 subunit of IL12/23, and human parathyroid hormone. Examples of small molecules whose activity is improved by multimerization include, for example, chemotherapeutic agents, cytotoxic molecules, dyes, and fluorophores, Specific examples of chemotherapeutic agents include mechlorethamine, chlorambucil, melphalan, daunorubicin, doxorubicin, epirubicin, idarubicin, mitoxantrone, valrubicin, paclitaxel, docitaxel, epothilones, etopiside, teniposide, tafluposide, azacitidine, azathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, and vinorelbine. Specific examples of cytotoxic molecules include methotrexate, cyclophosphamide, or azathioprine, mycophenolate, and cyclosporine A. CTLA-4, also known as CD152, is a member of the immunoglobulin superfamily that is expressed on T cells and transmits inhibitory signals to T cells. Like CD28, CTLA4 binds B7.1 and B7.2 (CD80 and CD86, respectively) on antigen presenting cells (APCs). However, unlike CD28, CTLA-4 transmits an inhibitory signal to the T cell instead of a co-stimulatory signal and binds with a higher affinity than does CD28. The increased affinity of CTLA-4 for the B7 molecules allows CTLA-4 to sequester B7 ligands from CD28 and antagonize CD28-dependant costimulation. CTLA-4 contains an extracellular domain, a transmembrane domain and a cytoplasmic tail. In accordance with the present invention, the entire CTLA-4 molecule may be included in the fusion protein or just the extracellular domain of CTLA-4 may be included in the fusion protein.

CTLA-4 has been an attractive pharmaceutical target. Since CTLA-4 binds with higher affinity to B7.1 and B7.2 molecules on APCs than does CD28, it has been investigated extensively as a potential therapy for autoimmune diseases. Fusion proteins of CTLA-4 and antibodies (CTLA4-Ig, abatacept) are commercially available for treatment of rheumatoid arthritis. Additionally, betacept, a second generation CTLA4-Ig fusion has recently been approved by the FDA for renal transplantation patients that are sensitized to Epstein Barr Virus.

Conversely, CTLA-4 antagonists also show great promise in the treatment of cancer as inhibitors of immune system tolerance. Antagonistic antibodies against CTLA-4 (ipilmumab) are being developed for this clinical indication.

CTLA-4 dimerization appears to be essential for biological activity. CTLA-4 does not undergo any detectable conformational change upon B7.2 binding (Schwartz, et al. (2001) “Structural basis for co-stimulation of the human CTLA-4/B7-2 complex,” Nature, 410(6828):604-8), and before ligation to B7 it exists as a nonfunctional covalent homodimer (Lindsey, et al. (1995) “Binding Stoichiometry of the Cytotoxic T Lymphocyte-associated Molecule-4 (CTLA-4)” J. Biol. Chem., 270(25):15417-15424). It has been shown that CTLA-4 dimerization/multimerization is hierarchically regulated by intermolecular disulfide bonding, N-linked glycosylation, and B7 ligand-driven dimerization. (Darlington, et al. (2005) “Hierarchical Regulation of CTLA-4 dimer-based lattice formation and its biological relevance for T cell inactivation,” J. Immunol., 175:996-1004). Therefore, the multimers of the current invention are particularly useful in enhancing the biological activity of CTLA-4 by creating dimers and higher ordered multimers of CTLA-4 proteins and peptides.

Programmed cell death-1 (PD-1) is an immunoreceptor belonging to the CD28/CTLA-4 family. PD-1 negatively regulates the antigen receptor signaling by recruiting protein tyrosine phosphatase, SHP-2, upon interaction with either of its two ligands, PDL-1 or PDL-2. (Okazaki and Honjo (2007) “PD-1 and PD-1 ligands: from discovery to clinical application,” International Immunol., 19(7):813-824).

PD-1 is a type I transmembrane glycoprotein comprising an IgV-type extracellular domain which shares homology with CTLA-4, CD28 and ICOS. PD-1 is expressed on peripheral T and B cells upon activation. PD-1 ligands PDL-1 and PDL-2 are also type I transmembrane glycoproteins composed of IgC and IgV-type extracellular domains, however, unlike PD-1, PDL-1 and PDL-2, comprise no intracellular signaling domains. (Okazaki and Honjo).

PD-1 dysregulation is associated with human autoimmune diseases such as lupus, rheumatoid arthritis, type I diabetes, multiple sclerosis, ankylosing spondylitis, myocardial infarction and allergy, while aberrant PDL-1 and PDL-2 are associated with many infectious diseases such as HIV, HCV, HBV, H. pylori infections. Antagonists of PD-1 are useful in the treatment of cancer and infectious disease, while agonists of PD-1 are useful in the treatment of autoimmunity, allergy and transplant rejection. Without being bound by theory, the PD-1, extracellular domain containing multimers of the present invention are useful in binding PDL-1 ligands without sending an intracellular signal, thereby acting as a PDL-1/PDL-2 sink and blocking PD-1 signaling. Therefore, the multimers of the current invention are particularly useful in blocking the biological activity of PD-1 by creating dimers and higher ordered multimers of PD-1 proteins and peptides which bind to PDL-1/PDL-2 without the associated cellular signaling.

IL-12 is a cytokine that is produced by dendritic cells, macrophages and certain B cells in response to antigenic stimulation. IL-12 functions in the differentiation of naïve T cells into Th0 cells that will eventually develop into Th1 cells in the continued presence of IL-12 since IL-12 also stimulates production of interferon gamma (IFNγ), and tumor necrosis factor alpha (TNFα), while decreasing IL-4 production, thus leading to differentiation of T cells into Th1 cells.

IL-23 is a cytokine produced by dendritic cells and macrophages in response to danger signals, including cell debris. IL-23 functions in directing memory T cells toward the Th17 phenotype. Therefore, while IL-12 mainly acts on naïve T cells, IL-23 acts predominantly on memory T cells.

Interleukin-12 (IL-12) is a heterodimer of the IL-12 p35 (IL-12α) and common p40 (IL-12β) subunits, while IL-23 is a heterodimer of the IL-23 p19 (IL-23α) and common p40 (IL-12β) subunits. Both heterodimeric IL-12 and IL-23 are agonistic for the IL-12 and IL-23 receptors, which in turn activate the transcription activator STAT4 to stimulate the production of IFNα. The naturally occurring homodimer of the common p40 subunit, on the other hand, is antagonistic of the IL-12 and IL-23 receptors. Therefore, while pharmaceutical companies have developed antibodies which are aimed at decreasing IL-12/23 receptor activity, these antibodies may also bind and remove from circulation, the naturally antagonistic p40/p40 homodimers. The present invention aims to increase the presence of p40/p40 homodimers and higher order multimers of p40/p40 in an effort to decrease proinflammatory IL-12/23 receptor signaling and dampen the Th1 immune response, which is particularly useful in patients with certain autoimmune diseases such as rheumatoid arthritis, type I diabetes and multiple sclerosis.

CD47 is a cell surface transmembrane Ig superfamily member and is an extracellular ligand for signal regulatory protein (SIRPα). Interactions between SIRPα and CD47 regulate immune cell functions such as neutrophil transmigration in response to inflammatory stimuli. (Liu, et al., (2004) “Peptide-Mediated Inhibition of Neutrophil Transmigration by Blocking cD47 Interactions with Signal Regulatory Protein α,” J. Immunol., 172:2278-2585).

Liu, et al., identified a novel function blocking peptide, CERVIGTGWWVRC (SEQ ID NO: 11) that mimics an epitope on CD47 and binds to SIRPα and is capable of inhibiting neutrophil migration into inflammatory sites. These peptides are referred to herein as “CERVIG peptides.” By CERVIG peptide is meant, any peptide containing the CERVIG residues and capable of binding SIRPα. SEQ ID NO: 11 is one example of a CERVIG peptide. Therefore, the multimers of the current invention, comprising CERVIG peptides, IgG2 hinge domains and optionally Fc domains are particularly useful in blocking the biological activity of CD47 by creating dimers and higher ordered multimers of the CERVIG peptides.

Antibodies including monoclonal antibodies can also be multimerized by the present invention. Examples of monoclonal antibodies include 3F8, 8H9, abagovomab, abciximab, adalimumab, adecatumumab, afelimomab, afutuzumab, alacizumab pegol, ALD518, alemtuzumab, altumomab pentetate, amatuximab, anatumomab mafenatox, anrukinzumab (IMA-638), apolizumab, arcitumomab, aselizumab, atinumab, atlizumab (tocilizumab), atorolimumab, bapineuzumab, basiliximab, bavituximab, bectumomab, belimumab, benralizumab, bertilimumab, besilesomab, bevacizumab, biciromab, bivatuzumab mertansine, blinatumomab, blosozumab, brentuximab vedotin, briakinumab, brodalumab, canakinumab, cantuzumab mertansine, cantuzumab ravtansine, capromab pendetide, carlumab, catumaxomab, CC49, cedelizumab, certolizumab pegol, cetuximab, Ch.14.18, citatuzumab bogatox, cixutumumab, clenoliximab, clivatuzumab tetraxetan, conatumumab, crenezumab, CR6261, dacetuzumab, daclizumab, dalotuzumab, daratumumab, denosumab, detumomab, dorlimomab aritox, drozitumab, ecromeximab, eculizumab, edobacomab, edrecolomab, efalizumab, efungumab, elotuzumab, elsilimomab, enavatuzumab, enlimomab pegol, enokizumab, ensituximab, epitumomab cituxetan, epratuzumab, erlizumab, ertumaxomab, etaracizumab, etrolizumab, exbivirumab, fanolesomab, faralimomab, farletuzumab, FBTA05, felvizumab, fezakinumab, ficlatuzumab, figitumumab, flanvotumab, fontolizumab, foralumab, foravirumab, fresolimumab, fulranumab, galiximab, ganitumab, gantenerumab, gavilimomab, gemtuzumab ozogamicin, gevokizumab, girentuximab, glembatumumab vedotin, golimumab, gomiliximab, GS6624, ibalizumab, ibritumomab tiuxetan, icrucumab, igovomab, imciromab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, lintuzumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-CD3, nacolomab tafenatox, namilumab, naptumomab estafenatox, narnatumab, natalizumab, nebacumab, necitumumab, nerelimomab, nimotuzumab, nofetumomab merpentan, ocrelizumab, odulimomab, ofatumumab, olaratumab, olokizumab, omalizumab, onartuzumab, oportuzumab monatox, oregovomab, otelixizumab, oxelumab, ozoralizumab, pagibaximab, palivizumab, panitumumab, panobacumab, pascolizumab, pateclizumab, pemtumomab, pertuzumab, pexelizumab, pintumomab, ponezumab, priliximab, pritumumab, PRO 140, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, siplizumab, sirukumab, solanezumab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, teneliximab, teplizumab, teprotumumab, TGN1412, ticilimumab (tremelimumab), tigatuzumab, TNX-650, tocilizumab (=atlizumab), toralizumab, tositumomab, tralokinumab, trastuzumab, TRBS07, tregalizumab, tremelimumab, tucotuzumab celmoleukin, tuvirumab, ublituximab, urelumab, urtoxazumab, ustekinumab, vapaliximab, vatelizumba, vedolizumab, veltuzumab, vepalimomab, vesencumab, visilizumab, volociximab, votumumab, zalutumumab, zanolimumab, ziralimumab, zolimomab aritox

The antibodies to be multimerized can be bispecific antibodies such as, for example blinatumomab. Blinatumomab has the structure scFv-ScFv, where one Fv is anti-CD19 and the other Fv is anti CD3A. The addition of an IgG2 hinge will result in a multimeric form of the antibody with multivalent binding to both epitopes, a useful feature for targeting low expressing antigens such as low expressing tumor-specific antigens on cancer cells. Additionally, multi-specific antibodies with multivalent binding to two or more epitopes can be created by the addition of an IgG2 hinge to a protein construct designed to express multiple antibodies.

Antigen binding domains of antibodies can be made using the variable portion of the heavy chain (V_{H}) linked to the variable portion of the light chain (V_{L}), forming an antigen binding site. (Holt, et al., (2003) “Domain antibodies: proteins for therapy,” Trends in Biotechnlogy, 21(11):484-490). The variable light chain can be linked to the variable heavy chain using one of many possible linker regions and may also contain the CH1 region of V_{H} or V_{L}. The V_{H} or V_{L} or both in tandem can be linked to the multimerizing IgG2 hinge domain and optionally to an Fc. Alternatively, the V_{H} or V_{L} may be co-expressed in the same cell and not fused as a chimeric protein. Alternatively, the V_{H} or V_{L} may be co-expressed in the same cell and each individually fused to the multimerizing IgG2 hinge domain as a chimeric protein. Multimerization of these antigen binding domains, herein referred to as “domain antibodies” are useful as soluble receptors, for example, to cytokines, such as TNF-α, IL-1, IL-12, IL-8, IFNα, IFNβ, IFNγ, IL-18, IL-27, and other proinflammatory mediators.

Multimerized domain antibodies are also useful as potent agonists of cell surface receptors, including, without limitation, hormonal receptors. Domain antibodies can also useful as potent antagonists of cell surface receptors, such as, for example, to a low expressing tumor receptor. Domain antibodies are also useful as diagnostic reagents, useful in methods such as, immunohistochemistry, flow cytometry, ELISA, ELISPOT, or any other assay where an antigen binding domain of an antibody is used. Single chain antibodies generated by placing the V_{L} domain and a linker on the N-terminus of the V_{H} domain of a human IgG1 monoclonal antibody (V_{L}-linker-V_{H}-CH1-CH2-CH3) (Wu, et al. (2001) “Multimerization of a chimeric anti-CD20 single-chain Fv-Fc fusion protein is mediated through variable domain exchange,” Protein Engineering 14(12):1025-1033) can also be linked to a multimerizing IgG2 hinge to form multimerized single chain antibodies such as:

a. IgG2 hinge-VL-linker-VH-IgG1 CH1-IgG1 Hinge-IgG1 CH2-IgG1 CH3
b. IgG2 hinge-VL-linker-VH-IgG1 CH1-IgG1 CH2-IgG1 CH3
c. VL-linker-VH-IgG2 hinge-IgG1 CH1-IgG1 Hinge-IgG1 CH2-IgG1 CH3
d. VL-linker-VH-IgG2 hinge-IgG1 CH1-IgG1 CH2-IgG1 CH3
e. VL-linker-VH-CH1-IgG2 Hinge-IgG Hinge-IgG1 CH2-IgG1 CH3
f. VL-linker-VH-CH1-IgG2 Hinge-IgG1 CH2-IgG1 CH3
g. VL-linker-VH-IgG1 CH1-IgG1 Hinge-IgG1 CH2-IgG1 CH3-IgG2 hinge
h. VL-linker-VH-IgG1 CH1-IgG1 CH2-IgG1 CH3-IgG2 hinge.

In addition to the domain antibodies discussed above, other small recombinant monoclonal antibody fragments and variants, including monovalent antibody fragments, such as Fab, scFv, diabodies, triabodies, minibodies and single domain antibodies can also be multimerized using the IgG2 hinge of the present invention. These fragments, like domain antibodies, retain the target specificity of whole monoclonal antibodies, but can be produced more echnomically and possess other unique and superior properties for a range of diagnostic and therapeutic applications. These monoclonal antibody fragments or variants can be directly linked to an IgG2 hinge with or without an Fc domain.

Additionally, the antibody-derived and non-Ig binding scaffolds summarized by Wurch, et al., (2008) “Development of novel protein scaffolds as alternatives to whole antibodies for imaging and therapy: status on discovery research and clinical validation,” Current Pharmaceutical Biotechnology, 23(9):1126-1136, can also be multimerized by the methods of the current invention.

Fully functional antibodies, termed nanobodies, lacking light chains, resembling those made by camels and llamas (Deffer, et al., (2009) African Journal of Biotechnology, 8(12):2645-2652) can also be multimerized by the IgG2 hinge of the present invention. These heavy-chain antibodies contain a single variable domain (VHH) and two constant domains (cCH2 and cCH3) in which the cloned and isolated VHH domain is a stable polypeptide harboring the full antigen-binding capacity of the original heavy-chain antibody. There is no cCH1 or light chain. This heavy chain antibody can be linked to the multimerizing IgG2 hinge domain to form a multimerizing single chain antibody as follows:

a. hIgG2 hinge-VHH-cHinge-cCH2-cCH3
b. hIgG2 hinge-VHH-cCH2-cCH3
c. VHH-hIgG2 hinge-cHinge-cCH2-cCH3
d. VHH-hIgG2 hinge-cCH2-cCH3
e. VHH-cHinge-cCH2-cCH3-hIgG2 hinge
f. VHH-cCH2-cCH3-hIgG2 hinge

Major histocompatibility complex (MHC) molecules expressed on the surface of antigen presenting cells bind antigen and present the antigen to T cells. MHC molecules, particularly MHC class I molecules, although MHC class II molecules can also be multimerized, are multimerized for use in tetramer assays. In certain embodiments, tetramer assays are used to detect the presence of antigen specific T-cells. In order for a T-cell to detect the peptide to which it is specific, it must recognize both the peptide and the MHC complex at the surface of a cell with which it comes into contact. Because the binding affinity of a T-cell receptor to MHC complexed with a peptide is so low, this has historically been a challenging problem. An earlier solution was realized by creating a tetramer of MHC molecules each presenting an identical peptidic antigen which increases the avidity of the binding. Multimerizing the MHC-antigen complex with the IgG2 hinge will further increase binding affinity and avidity. These compounds can be labeled for use in detection, for example with a dye or fluorophore. Similar compounds comprising the biotin-streptavidin MHC tetramers can also be used.

The important role of multivalent carbohydrate derivatives in glycobiology is well described. (R. Roy, Topics Curr Chem 1997, 187; 241-274; M. Mammen, S. K. Choi, G. M. Whitesides, Angew. Chem 1998, 110; 2908-2953).

Linear or branched oligonucleotide multimers are useful as amplifiers in biochemical assays U.S. Pat. No. 5,124,246)

Higher fatty acids can form associative multimers by hydrogen binding as a result of the presence of negatively polarized oxygen atom from the carbonyl group and the positively polarized hydrogen atom from the carboxyl group (Preparative Layer Chromatography, Teresa Kowalska and Joseph Sherma). Multimers of certain fatty acids may be therapeutically useful, alone or in combination with proteins. High-density lipoprotein (HDL) is positively associated with a decreased risk of coronary heart disease (CHD). HDL is composed of 4 apolipoproteins per particle. HDL itself is heterogeneous. HDL may be composed of apo A-I and apo A-II or of apo A-I alone. HDL2 is usually made up only of apo A-I, while HDL3 contains a combination of apo A-I and apo A-II. HDL particles that are less dense than HDL2 are rich in apo E. A fusion protein of the IgG2 hinge and either a fatty acid, or combination of fatty acids, or one or more apolipoproteins will create multimers of that fatty acid or apolipoprotein(s). These multimers may be therapeutically useful, for example in reversing cholesterol transport, improving atherosclerosis, decreasing primary or secondary myocardial or stroke risk, or treating diseases of lipid-rich organs such as brain, including Alzheimer's Disease and Parkinson's Disease.

Improved blood clotting is sometimes needed, for example in surgical situations. Von Willebrand factor is a protein crucial for blood clotting (Science 324, 1330-1334). Endothelial cells secrete von Willebrand Factor as very large multimers which is then cleaved quickly to smaller multimers by available metalloproteases such as ADAMTS13 (Nat. Med 15 (7); 738). Smaller multimers of von Willebrand factor are worse than the larger multimers at promoting blood clots. A fusion protein of the IgG2 hinge and von Willebrand factor will increase the multimer size and increase functionality.

### Fc Domain

As used herein, “Fc domain” or “Immunoglobulin Fc domain” describes the minimum region (in the context of a larger polypeptide) or smallest protein folded structure (in the context of an isolated protein) that can bind to or be bound by an Fc receptor (FcR). In both an Fc fragment and an Fc partial fragment, the Fc domain is the minimum binding region that allows binding of the molecule to an Fc receptor. While an Fc domain can be limited to a discrete polypeptide that is bound by an Fc receptor, it will also be clear that an Fc domain can be a part or all of an Fc fragment, as well as part or all of an Fc partial fragment. When the term “Fc domains” is used in this invention it will be recognized by a skilled artisan as meaning more than one Fc domain. An Fc domain is comprised of two Fc domain monomers. As further defined herein, when two such Fc domain monomers associate to form a homodimer, the resulting Fc domain has Fc receptor binding activity. Thus an Fc domain is a homodimeric structure that can bind an Fc receptor.

The specific CH1, CH2, CH3 and CH4 domains and hinge regions that comprise the Fc domain monomers of the fusion proteins of the present invention may be independently selected, both in terms of the immunoglobulin subclass, as well as in the organism, from which they are derived. Accordingly, the fusion proteins disclosed herein may comprise Fc domain monomers and partial Fc domain monomers that independently come from various immunoglobulin types such as human IgG1, IgG2, IgG3, IgG4, IgA1, IgA2, IgD, IgE, and IgM, mouse IgG2a, or dog IgA or IgB. Similarly each Fc domain monomer and partial Fc domain monomer may be derived from various species, preferably a mammalian species, including non-human primates (e.g., monkeys, baboons, and chimpanzees), humans, murine, rattus, bovine, equine, feline, canine, porcine, rabbits, goats, deer, sheep, ferrets, gerbils, guinea pigs, hamsters, bats, birds (e.g., chickens, turkeys, and ducks), fish and reptiles to produce species-specific or chimeric fusion proteins.

The individual Fc domain monomers and partial Fc domain monomers may also be humanized. One of skill in the art will realize that different Fc domains and partial Fc domains will provide different types of functionalities. For example, FcγRs bind specifically to IgG immunoglobulins and not well to other classes of immunoglobulins.

“Capable of specifically binding to a FcγR” as used herein refers to binding to an FcγR. Specific binding is generally defined as the amount of labeled ligand which is displaceable by a subsequent excess of unlabeled ligand in a binding assay. However, this does not exclude other means of assessing specific binding which are well established in the art (e.g., Mendel C M, Mendel D B, ‘Non-specific’ binding. The problem and a solution. Biochem J. 1985 May 15; 228(1):269-72). Specific binding may be measured in a variety of ways well known in the art such as surface plasmon resonance (SPR) technology (commercially available through BIACORE®) or biolayer interferometry (commercially available through ForteBio®) to characterize both association and dissociation constants of the fusion proteins (Asian K. Lakowicz J R, Geddes C. Plasmon light scattering in biology and medicine: new sensing approaches, visions and perspectives. Current Opinion in Chemical Biology 2005, 9:538-544). The fusion proteins of the present invention are designed in such a way as to include Fc domain monomers, however the Fc domain monomer to be included in the fusion protein is selected for poor binding to Fc gamma receptors. By “poor binding to Fc gamma receptors” is meant that the Fc domain binds with relatively weaker affinity than an Fc gamma receptor that binds with higher affinity. For example. IgG2 and IgG4 naturally bind poorly to Fc gamma receptors and therefore these isotypes are particularly useful in the present invention.

Alternatively, an isotype such as IgG1, which under normal circumstances binds to Fc gamma receptors with higher affinity may be mutated or otherwise modified to decrease Fc binding affinity. These mutations and modifications are described below in more detail. Without being bound by theory, it is thought that a fusion protein containing an Fc domain that does not bind well to Fc gamma receptors retains useful drug characteristics relative to fusion proteins not containing the Fc domain including increased serum half life and more efficient manufacturing purification. Therefore, a fusion protein whose primary sequence is comprised of an IgG2 hinge will create multimers that will have important clinical and non-clinical utility, independent of their binding to Fc gamma receptors.

The present invention also encompasses fusion proteins comprising Fc domains and Fc partial domains having amino acids that differ from the naturally-occurring amino acid sequences of the Fc domain. Preferred Fc domains for inclusion in the fusion proteins of the present invention have poor specific binding affinity to either a holo-Fcγ receptor or a soluble extracellular domain portion of an FcγR. Primary amino acid sequences and X-ray crystallography structures of numerous Fc domains and Fc domain monomers are available in the art. See, e.g., Woof J M, Burton D R. Human antibody-Fc receptor interactions illuminated by crystal structures. Nat Rev Immunol. 2004 February; 4(2):89-99. Representative Fc domains with Fcγ receptor binding capacity include the Fc domains from human IgG1. These native sequences have been subjected to extensive structure-function analysis including site directed mutagenesis mapping of functional sequences. Based on these prior structure-function studies and the available crystallography data, one of skill in the art may design functional Fc domain sequence variants that diminish the Fc domain's FcγR receptor binding capacity but which retain functional utility, such as creating a more efficiently purified fusion protein or a fusion protein with a longer half-life than the same protein lacking the Fc domain or partial domain.

The amino acid changes may be found throughout the sequence of the Fc domain, or be isolated to particular Fc partial domains that comprise the Fc domain. The functional variants of the Fc domain used in the fusion proteins of the present invention will have at least about 50%, 60%, 70%, 80%, 90%, 95%, 96%, 97%, 98% or 99% sequence identity to a native Fc domain. Similarly, the functional variants of the Fc partial domains used in the fusion proteins of the present invention will have at least about 50%, 60%, 70%, 80%, 90%, 95%, 96%, 97%, 98% or 99% sequence identity to a native Fc partial domain.

The skilled artisan will appreciate that the present invention further encompasses the use of functional variants of Fc domain monomers in the construction of Fc fragment monomers, Fc partial fragment monomers, and fusion proteins of the present invention. The functional variants of the Fc domain monomers will have at least about 50%, 60%, 70%, 80%, 90%. 95%, 96%, 97%, 98% or 99% sequence identity to a native Fc domain monomer sequence.

The amino acid changes decrease or prevent altogether the binding affinity of the fusion protein to the Fcγ receptor. Preferably such amino acid changes will be conservative amino acid substitutions, however, such changes include deletions, additions and other substitutions. Conservative amino acid substitutions typically include changes within the following groups: glycine and alanine; valine, isoleucine, and leucine; aspartic acid and glutamic acid; asparagine, glutamine, serine and threonine; lysine, histidine and arginine; and phenylalanine and tyrosine. Additionally, the amino acid change may enhance multimerization frequency, extent, percentage, or strength, for example by the addition of cysteine residues.

The amino acid changes may be naturally occurring amino acid changes resulting in Fc domain polymorphisms, or the amino acid changes may be introduced, for example by site directed mutagenesis. The amino acid changes can occur anywhere within the Fc domain so long as the Fc domain retains the desired biological activity. In a preferred embodiment, the polymorphism or mutation leads to decreases receptor binding. The polymorphism/mutation preferably occurs at one or more of amino acid positions 233, 234, 235, 236, 238, 239, 265, 269, 270, 292, 293, 295, 296, 297, 303, 327, 329, 338, 376, and/or 414 according to the EU index as in Kabat et al., Sequences of Proteins of Immunological Interest, 5th Ed. Public Health Service, National Institutes of Health, Bethesda, Md. (1991). Specific polymorphisms/mutations in these amino acid positions are well known in the art and can be found, for example in Sheilds, et al. (2001) J. Biol. Chem. 276(9):6591-6604.

In a preferred embodiment, the polymorphism/mutation contains one or more amino acid substitutions of positions 233, 234, 235, 236, 238, 239, 265, 269, 270, 292, 293, 295, 296, 297, 303, 327, 329, 338, 376, and/or 414 of IgG1 Fc. In a further embodiment, the polymorphism/mutation contains two or more amino acid substitutions of positions 233, 234, 235, 236, 238, 239, 265, 269, 270, 292, 293, 295, 296, 297, 303, 327, 329, 338, 376, and/or 414 of IgG1 Fc. In a further embodiment, the polymorphism/mutation contains three or more amino acid substitutions of positions 233, 234, 235, 236, 238, 239, 265, 269, 270, 292, 293, 295, 296, 297, 303, 327, 329, 338, 376, and/or 414 of IgG1 Fc. In a further embodiment, the polymorphism/mutation contains more than three amino acid substitutions of positions 233, 234, 235, 236, 238, 239, 265, 269, 270, 292, 293, 295, 296, 297, 303, 327, 329, 338, 376, and/or 414 of IgG1 Fc.

The term “functional variant” as used herein refers to a sequence related by homology to a reference sequence which is capable of mediating the same biological effects as the reference sequence (when a polypeptide), or which encodes a polypeptide that is capable of mediating the same biological effects as a polypeptide encoded by the reference sequence (when a polynucleotide). Functional sequence variants include both polynucleotides and polypeptides. Sequence identity is assessed generally using BLAST 2.0 (Basic Local Alignment Search Tool), operating with the default parameters: Filter-On, Scoring Matrix-BLOSUM62, Word Size-3, E value-10, Gap Costs-11,1 and Alignments-50.

In addition to the amino acid sequence composition of native Fc domains, the carbohydrate content of the Fc domain is known to play an important role on Fc domain structure and binding interactions with FcγR. See, e.g., Robert L. Shields, et al. Lack of Fucose on Human IgG1 N-Linked Oligosaccharide Improves Binding to Human FcγRIII and Antibody-dependent Cellular Toxicity. J. Biol. Chem., July 2002; 277: 26733-2674; Ann Wright and Sherie L. Morrison. Effect of C2-Associated Carbohydrate Structure on Ig Effector Function Studies with Chimeric Mouse-Human IgG1 Antibodies in Glycosylation Mutants of Chinese Hamster Ovary Cells. J. Immunol, April 1998; 160: 3393-3402. Carbohydrate content may be controlled using, for example, particular protein expression systems including particular cell lines or in vitro enzymatic modification. Thus, the present invention includes fusion proteins comprising Fc domains with the native carbohydrate content of holo-antibody from which the domains were obtained, as well as those fusion proteins with an altered carbohydrate content. In another embodiment, multimer components of the fusion protein are characterized by a different glycosylation pattern compared with the homodimer component of the same fusion protein. In a preferred embodiment, the fusion protein is enriched for homodimers and multimers comprising a glycosylation pattern that decreases Fc receptor binding. In a particular embodiment, the Fc domain is hyperfucosylated, demannosylated or hemi-glycosylated, thus resulting in decreased binding to Fc receptors (Yamme-Ohnuki and Sato (2009) “Production of Therapeutic Antibodies with Controlled Fucosylation,” mAbs, 1:3, 230-236).

The present invention also encompasses fusion proteins comprising Fc domains which comprise antigen binding-sites in the structural loops of the Fc domains, such as those described by Wozniak-Knopp, et al. (2010) “Introducing antigen-binding sites in structural loops of immunoglobin constant domains: Fc fragments with engineered HER2/neu-binding sites and antibody properties. In these constructs, antigen binding sites are introduced in the loop regions, particularly the loop regions located at the C-terminal tip of the Fc CH3 domain which includes loops AB, CD and EF. These engineered Fcs can not only bind to antigen through the engineered antigen binding site, but also retain the ability to elicit effector functions via binding to Fcγ receptors, complement and FcRn. These engineered Fc can comprise antigen binding domains that bind any antigen. For example, the Fc can be engineered to comprise an antigen binding site that binds the same antigens as the monoclonal antibodies discussed above. In one embodiment, the Fc is engineered to express a Her2/neu binding site. In a further embodiment, the Fc engineered to express a Her2/neu binding site (SEQ ID NO: 18) is fused to an IgG2 hinge to mediate multimerization of the engineered Fc. The IgG2 hinge can be fused to C terminus of the engineered Fc (SEQ ID NO: 19) or the N terminus of the engineered Fc (SEQ ID NO: 20). Additionally, the fusion protein may comprise an Fc CH2 and CH3 domain between the IgG2 hinge and the engineered FC (IgG2 hinge-CH2-CH3-engineered Fc) (SEQ ID NO: 21). These multimerized compounds will have a high avidity for both the antigen, e.g. Her2/neu as well as FcγRIIIa which will increase the tumor killing potency of the compounds.

### Fusion Proteins

As used herein, the term “fusion protein” means any single, contiguous peptide molecule that contains at least the IgG2 hinge of SEQ ID NO: 1 and a peptide, protein, nucleic acid, fatty acid, or small molecule whose activity is increased by multimerization. The fusion protein of the present invention may also include an immunoglobulin Fc domain monomer which binds with decreased affinity, or not at all, to Fc receptors. Furthermore, the fusion proteins of the present invention also refers to a protein that, when associated with at least a second fusion protein, forms a dimeric or multimeric structure comprising at least two peptides, proteins, nucleic acids, fatty acids, or small molecules whose activity is increased by multimerization.

The regions of the fusion proteins, the IgG2 hinge, the peptide, protein, nucleic acids, fatty acids, or small molecule to be multimerized and the immunoglobulin Fc domain monomer, may be arranged from amino terminal to carboxy terminal of successive regions of the fusion protein. The regions may be directly linked to each other or linked using small regions of amino acid linker residues. Fusion proteins of the present invention comprise the amino terminus of the peptide or protein, linked to the carboxy terminus of the IgG2 hinge or the amino terminus of the IgG2 hinge linked to the carboxy terminus of the peptide, protein. With respect to small molecules, nucleic acids and fatty acids, the fusion protein may comprise a small molecule, nucleic acid or fatty acid linked to the carboxy terminus of the IgG2 hinge. Conversely, the fusion protein may comprise a small molecule, nucleic acid or fatty acid linked to the amino terminus of the IgG2 hinge. For example, the fusion proteins of the present invention may include:
Protein/peptide/small molecule/nucleic acid/fatty acid-IgG2 hinge
IgG2 hinge-protein/peptide/small molecule/nucleic acid/fat
   Fusion proteins of the present invention also comprise small molecules or fatty acids linked to the IgG2 hinge. For example, the small molecule, fatty acid, or nucleic acid may be
Small molecule/nucleic acid/fatty acid-IgG2 hinge
IgG2 hinge-small molecule/nucleic acid/fatty acid
   Alternatively the small molecule, nucleic acid, or fatty acid may be linked to the IgG2 hinge through a linker or through a sugar bound to the IgG2 hinge.

In one embodiment, the peptide/protein/small molecule/nucleic acid/fatty acid is a IL-12 p40 subunit. In another embodiment, the peptide/protein/small molecule/nucleic acid/fatty acid is a CTLA4 protein or CTLA-4 extracellular domain peptide. In a further embodiment, the peptide/protein/small molecule/nucleic acid/fatty acid is a PD-1 protein or PD-1 extracellular domain peptide. In a further embodiment, the peptide/protein/small molecule/nucleic acid/fatty acid is a CERVIG peptide. In a further embodiment the exemplified fusion proteins contain a CTLA4 protein or CTLA4 extracellular domain peptide and also contain a B7 molecule. Examples of such molecules include:
CTLA4-IgG2 hinge
IgG2 hinge-CTLA4
B7-IgG2 hinge
IgG2 hinge-B7
IgG2 hinge-CTLA4-B7
B7-CTLA4-IgG2 hinge
CTLA4-B7-IgG2 hinge
IgG2 hinge-B7-CTLA4
IgG2 hinge-CTLA4-B7

In one embodiment the B7 is B7.1. In another embodiment, the B7 is B7.2.

Additional fusion proteins of the present invention comprise the amino terminus of the peptide, protein, small molecule, nucleic acid or fatty acid linked to the carboxy terminus of the IgG2 hinge which is in turn linked via the amino terminus of the IgG2 hinge to the carboxy terminus of an immunoglobulin Fc domain monomer, for example, Fc-IgG2 hinge-peptide protein small molecule/nucleic acid/fatty acid

The fusion proteins of the present invention may also include:
Protein/peptide/small molecule/nucleic acid/fatty acid-IgG2 hinge-Fc
Fc-Protein/peptide/small molecule/nucleic acid/fatty acid-IgG2 hinge
IgG2 hinge-Fc-peptide/protein/small molecule/nucleic acid/fatty acid
Protein/peptide/small molecule/nucleic acid/fatty acid-Fc-IgG2 hinge
IgG2 hinge-peptide/protein/small molecule/nucleic acid/fatty acid-Fc

In one embodiment, the peptide/protein/small molecule/nucleic acid/fatty acid is a IL-12 p40 subunit. In another embodiment, the peptide/protein/small molecule/nucleic acid/fatty acid is a CTLA4 protein or CTLA-4 extracellular domain peptide. In a further embodiment, the peptide/protein/small molecule/nucleic acid/fatty acid is a PD-1 protein or PD-1 extracellular domain peptide. In a further embodiment, the peptide/protein/small molecule/nucleic acid/fatty acid is a CERVIG peptide SEQ ID NO: 11. In a further embodiment the exemplified fusion proteins contain a CTLA4 protein or peptide and also contain a B7 molecule. Examples of fusion proteins containing B7 molecules include:
CTLA4-IgG2 hinge-Fc
CTLA4-Fc-IgG2 hinge
Fc-CTLA4-IgG2 hinge
IgG2 hinge-CTLA4-Fc
Fc-IgG2 hinge-CTLA4
IgG2 hinge-Fc-CTLA4
B7-IgG2 hinge-Fc
B7-Fc-IgG2 hinge
Fc-B7-IgG2 hinge
IgG2 hinge-B7-Fc
Fc-IgG2 hinge-B7
IgG2 hinge-Fc-B7
B7-CTLA4-IgG2 hinge-Fc
Fc-B7-CTLA4-IgG2 hinge
B7-CTLA4-Fc-IgG2 hinge
CTLA4-B7-IgG2 hinge-Fc
Fc-CTLA4-B7-IgG2 hinge
CTLA4-B7-Fc-IgG2 hinge
IgG2 hinge-B7-CTLA4-Fc
Fc-IgG2 hinge-B7-CTLA4
IgG2 hinge-Fc-B7-CTLA4
IgG2 hinge-CTLA4-B7-Fc
Fc-IgG2 hinge-CTLA4-B7
IgG2 hinge-Fc-CTLA4-B7

In one embodiment the B7 is B7.1. In another embodiment, the B7 is B7.2.

In another embodiment, the peptide/protein/small molecule/nucleic acid/fatty acid is a IL-12 p40 subunit.
IgG2 hinge-IL-12 p40
IL-12 p40-IgG2 hinge
IgG2 hinge-Fc domain-IL-12 p40
IgG2 hinge-IL-12 p40-Fc domain
Fc domain-IL-12 p40-IgG2 hinge
IL-12 p40-Fc domain-IgG2 hinge
Fc domain-IgG2 hinge-IL-12 p40
IL-12 p40-IgG2 hinge-Fc domain

In a further embodiment, the peptide/protein/small molecule/nucleic acid/fatty acid is a PD-1 protein or PD-1 extracellular domain peptide.
IgG2 hinge-PD-1
PD-1-IgG2 hinge
IgG2 hinge-Fc domain-PD-1
IgG2 hinge-PD-1-Fc domain
Fc domain-PD-1-IgG2 hinge
PD-1-Fc domain-IgG2 hinge
Fc domain-IgG2 hinge-PD-1
PD-1-IgG2 hinge-Fc domain

In a further embodiment, the peptide/protein/small molecule/nucleic acid/fatty acid is a CERVIG peptide.
IgG2 hinge-CERVIG
CERVIG-IgG2 hinge
IgG2 hinge-Fc domain-CERVIG
IgG2 hinge-CERVIG-Fc domain
Fc domain-CERVIG-IgG2 hinge
CERVIG-Fc domain-IgG2 hinge
Fc domain-IgG2 hinge-CERVIG
CERVIG-IgG2 hinge-Fc domain

In another embodiment, the peptide/protein/small molecule/nucleic acid/fatty acid is Von Willebrand factor. Examples of fusion proteins containing Von Willebrand Factor molecules include:
IgG2 hinge-VWF
VWF-IgG2 hinge
IgG2 hinge-Fc domain-VWF
IgG2 hinge-VWF-Fc domain
Fc domain-VWF-IgG2 hinge
VWF-Fc domain-IgG2 hinge
Fc domain-IgG2 hinge-VWF
VWF-IgG2 hinge-Fc domain

In another embodiment, the peptide/protein/small molecule/nucleic acid/fatty acid is an MHC molecule. In this embodiment, MHC molecules are multimerized to form multimers such as MHC tetramers. Examples of fusion proteins containing MHC molecules include:
fluorescent tag-MHC-antigen-IgG2 hinge
fluorescent tag-IgG2 hinge-MHC-antigen
MHC-antigen-IgG2 hinge
IgG2 hinge-MHC-antigen
IgG2hinge-MHC-antigen-biotinylated tetramer unit

In another embodiment, small peptides are fused to the IgG2 hinge, with or without an Fc, in order to form multimers that increase the affinity and avidity of binding of the small peptides to their target because of multivalent binding. Small peptides often have very short clinical half lives which can be improved through the addition of Fc. In one embodiment of this approach, a 21 mer peptide with the sequence LGASWHRPDKCCLGYQKRPLP (SEQ ID NO: 2) is a peptide antagonist of CXCR4 (Zhou et al Biochemistry 2000 39(13) pp 3782). The peptide has been demonstrated to prevent CXCR4 signaling and CXCR4-mediated entry of HIV into CD4 cells. By adding the IgG2 hinge, the peptide will multimerize and will bind with greater affinity and avidity. By adding the Fc, the half life of the multimerized compound is increased. Therapeutic uses include treatment of HIV disease and immune disorders. Examples of fusion proteins containing the LGASWHRPDKCCLGYQKRPLP (SEQ ID NO: 2) peptide include:

(SEQ ID NO: 2)
LGASWHRPDKCCLGYQKRPLP-IgG2 hinge-IgG4 Fc
(SEQ ID NO: 2)
LGASWHRPDKCCLGYQKRPLP-IgG4 Fc-IgG2 hinge
(SEQ ID NO: 2)
IgG4 Fc-LGASWHRPDKCCLGYQKRPLP-IgG2 hinge
(SEQ ID NO: 2)
IgG4 Fc-IgG2 hinge-LGASWHRPDKCCLGYQKRPLP
(SEQ ID NO: 2)
IgG2 hinge-IgG4 Fc-LGASWHRPDKCCLGYQKRPLP
(SEQ ID NO: 2)
IgG2 hinge-LGASWHRPDKCCLGYQKRPLP-IgG4 Fc
(SEQ ID NO: 2)
LGASWHRPDKCCLGYQKRPLP-IgG2 hinge-IgG2 CH2-IgG2 CH3
(SEQ ID NO: 2)
LGASWHRPDKCCLGYQKRPLP-IgG2 CH2-IgG2 CH3-IgG2 hinge
(SEQ ID NO: 2)
IgG2 CH2-IgG2 CH3-LGASWHRPDKCCLGYQKRPLP-IgG2 hinge
(SEQ ID NO: 2)
IgG2 CH2-IgG2 CH3-IgG2 hinge LGASWHRPDKCCLGYQKRPLP
(SEQ ID NO: 2)
IgG2 hinge-IgG2 CH2-IgG2 CH3-LGASWHRPDKCCLGYQKRPLP
(SEQ ID NO: 2)
IgG2 hinge-LGASWHRPDKCCLGYQKRPLP-IgG2 CH2-IgG2 CH3

In another similar embodiment, Nakamura et al (Journal of Biotechnology 2005; 116 (3); 211-219) have identified a peptide which mimics EGF binding to EGFR and inhibits mitogenesis and EGFR signaling. This small peptide can be linked to IgG2 hinge and optionally to Fc as described above. This will be useful as an anticancer agent.

In another embodiment, Maruta et al (Cancer Gene Ther. 2002. 9(6); 543-52) have identified the small peptide MQLPLAT (SEQ ID NO: 3) that binds FGF receptor-expressing cells. MQLPLAT (SEQ ID NO: 3) binds to and accumulates in cancer cells relative to normal. They are not characterized as antagonist or agonist. This small peptide can be linked to IgG2 hinge and optionally to Fc as described above. This will be useful as an anticancer agent and could be used for specific delivery of a toxic compound to target cells by a compound. Examples of fusion proteins containing the MQLPLAT (SEQ ID NO: 3) peptide include:
IgG2 hinge-IgG4 Fc-FGF binding peptide-peptide toxin
IgG2 hinge-IgG4 Fc-FGF binding peptide-small molecule toxin
IgG4 Fc-FGF binding peptide-peptide toxin-IgG2 hinge
IgG4 Fc-FGF binding peptide-small molecule toxin-IgG2 hinge
FGF binding peptide-peptide toxin-IgG2 hinge-IgG4 Fc
FGF binding peptide-small molecule toxin-IgG2 hinge-IgG4 Fc
IgG2 hinge-IgG2 CH2-IgG2 CH3-FGF binding peptide-peptide toxin
IgG2 hinge-IgG2 CH2-IgG2 CH3-FGF binding peptide-small molecule toxin
IgG2 CH2-IgG2 CH3-FGF binding peptide-peptide toxin-IgG2 hinge
IgG2 CH2-IgG2 CH3-FGF binding peptide-small molecule toxin-IgG2 hinge
FGF binding peptide-peptide toxin-IgG2 hinge-IgG2 CH2-IgG2 CH3
FGF binding peptide-small molecule toxin-IgG2 hinge-IgG2 CH2-IgG2 CH3

In another embodiment, Ruff et al (FEBS letters 1987: 211(1); 17-22) have identified an octa peptide ASTTTNYT (SEQ ID NO: 4) that blocks CD4 receptor binding by HIV. This small peptide can be linked to IgG2 hinge and optionally to Fc as described above. This will be useful as an antiviral.

In another embodiment, Noberini et al (PlosOne. 2011. 6(12) e28611) have identified a 15 mer peptide called TNYL-RAW that targets the EphB4 receptor and inhibits angiogenesis through disruption of EphB4-ephrin-B2 interactions. This small peptide can be linked to IgG2 hinge and optionally to Fc as described above. This will be useful as an oncolytic agent. In another embodiment, Holt et al., (Trends in Biotechnology. 21(11)(2003) and others have described the ability to generate binding domains using the variable portion of the heavy chain (VH) linked to the variable portion of the light chain (VL), forming an epitope binding site. The variable light chain can be linked to the variable heavy chain using one of many possible linker regions and may contain the CH1 region of VH or VL. The VH or VL or both can be linked to the multimerizing IgG2 hinge domain and optionally to Fc as described above. Alternatively the VH or VL may not be directly fused as a chimeric protein but may instead be co-expressed in the same cell. In a further embodiment the co-expressed VL is also linked to an IgG2 hinge region. In a further embodiment, the VH is not linked to a CH1 so that the co-expressed peptides comprise VH-IgG2 hinge and VL.

Holliger and Hudson (Nat Biotechnol. 2005 September; 23(9):1126-36) summarize a range of small recombinant fragments that are variants of a monoclonal antibody, including monovalent antibody fragments such as Fab, scFv and engineered variants; diabodies, triabodies, minibodies and single-domain antibodies. These fragments retain the targeting specificity of whole monoclonal antibodies but can be produced more economically and possess other unique and superior properties for a range of diagnostic and therapeutic applications. Each of these can similarly be multimerized by inclusion of the IgG2 hinge domain as disclosed above.

Wurch et al (Current Pharmaceutical Biotechnology, 2008, 9, 502-509) summarize about fifty different antibody-derived and non-Ig scaffolds that have been discovered and documented, including scaffolds of the IgG superfamily, loop-containing or highly structured protein scaffolds that provide a rigid core structure suitable for grafting loops, oligomeric protein scaffolds allowing the incorporation of variable loops in a favorable 3D configuration, and carrier proteins that display a single binding interface. Each of these can similarly be multimerized by inclusion of the IgG2 hinge domain as disclosed above.

In another embodiment, Wu et al 2001 Protein Engineering Vol 14 no 12 pp 1025-1033 have demonstrated the ability to generate single chain antibodies by placing the V_{L} domain and a linker on the N-terminus of the V_{H} domain of a human IgG1 monoclonal antibody (V_{L}-linker-V_{H}-CH1-CH2-CH3).

In another embodiment, Deffar et al (African Journal of Biotechnology Vol. 8 (12), pp. 2645-2652, 17 June, 2009) have demonstrated that camels and llamas possess fully functional antibodies that lack light chains. These heavy-chain antibodies contain a single variable domain (VHH) and two constant domains (cCH2 and cCH3) in which the cloned and isolated VHH domain is a stable polypeptide harboring the full antigen-binding capacity of the original heavy-chain antibody. There is no cCH1 or light chain. The authors call these heavy chain antibodies nanobodies. This heavy chain antibody can be linked to the multimerizing IgG2 hinge domain to form a multimerizing single chain antibody as follows:
hIgG2 hinge-V_{HH}-cHinge-cCH2-cCH3
hIgG2 hinge-V_{HH}-cCH2-cCH3
V_{HH}-hIgG2 hinge-cHinge-cCH2-cCH3
V_{HH}-hIgG2 hinge-cCH2-cCH3
V_{HH}-cHinge-cCH2-cCH3-hIgG2 hinge
V_{HH}-cCH2-cCH3-hIgG2 hinge

The immunoglobulin Fc domain is any Fc domain or fragment that binds with lower affinity to Fc gamma receptors than does native IgG1, but which also retains other beneficial features including, the retained ability to bind to Protein A or Protein G affinity columns or preferred binding to the neonatal FcN receptor. The Fc domains may be naturally occurring, may be comprised of naturally occurring components in non-natural combination, or may be comprised of naturally occurring and non-natural components. Examples of such Fc domains include:
IgG2 hinge-IgG2 CH2-IgG2 CH3
IgG2 hinge-IgG3 CH2-IgG3 CH3
IgG2 hinge-IgG3 CH2-IgG2 CH3
IgG2 hinge-IgG2 CH2-IgG3 CH3
IgG4 hinge-IgG4 CH2-IgG4 CH3
IgG2 CH2-IgG2 CH3
IgG4 CH2-IgG4 CH3
IgG4 CH2-IgG2 CH3
IgG2 CH2-IgG4 CH3
Hemiglycosylated IgG1 hinge-IgG1 CH2-IgG1 CH3
Hemiglycosylated IgG1 CH2-IgG1 CH3
Hemiglycosylated IgG3 hinge-IgG3 CH2-IgG3 CH3
Hemiglycosylated IgG3 CH2-IgG3 CH3
IgG1 hinge-IgG2 CH2-IgG3 CH3
IgG1 hinge-IgG1 CH2 N297A-IgG1 CH3
IgG1 hinge-IgG1 CH2 D265A-IgG1 CH3

Alternatively, the immunoglobulin Fc domain is an Fc domain or fragment that has been engineered to contain an antigen binding site. These fusion proteins are useful in the treatment of inflammatory disease, autoimmune disease and cancer. For example, an Fc engineered to express a Her2/neu antigen binding site will be useful in the treatment of breast cancer. Examples of fusion proteins comprising such Fc domains are:
Engineered Fc-IgG2 hinge
IgG2 hinge-Engineered Fc
IgG2 hinge-CH2-CH3-Engineered Fc

### Pharmaceutical Compositions

Administration of the fusion protein compositions described herein will be via any common or uncommon route, orally, parenterally, or topically. Exemplary routes include, but are not limited to oral, nasal, buccal, rectal, vaginal, ophthalmic, subcutaneous, intramuscular, intraperitoneal, intravenous, intraarterial, intratumoral, spinal, intrathecal, intra-articular, intra-arterial, sub-arachnoid, sublingual, oral mucosal, bronchial, lymphatic, intra-uterine, subcutaneous, intratumor, integrated on an implantable device such as a suture or in an implantable device such as an implantable polymer, intradural, intracortical, or dermal. Such compositions would normally be administered as pharmaceutically acceptable compositions as described herein. In a preferred embodiment the isolated fusion protein is administered intravenously or subcutaneously.

The term “pharmaceutically acceptable carrier” as used herein includes any and all solvents, dispersion media, coatings, antibacterial and antifungal agents, isotonic and absorption delaying agents, hydrogels, and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The fusion protein compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Sterile injectable solutions are prepared by incorporating the fusion proteins in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Further, one embodiment is a fusion protein composition suitable for oral administration and is provided in a pharmaceutically acceptable carrier with or without an inert diluent. The carrier should be assimilable or edible and includes liquid, semi-solid, i.e., pastes, or solid carriers. Except insofar as any conventional media, agent, diluent or carrier is detrimental to the recipient or to the therapeutic effectiveness of a fusion protein preparation contained therein, its use in an orally administrable a fusion protein composition for use in practicing the methods of the present invention is appropriate. Examples of carriers or diluents include fats, oils, water, saline solutions, lipids, liposomes, resins, binders, fillers and the like, or combinations thereof. The term “oral administration” as used herein includes oral, buccal, enteral or intragastric administration.

In one embodiment, the fusion protein composition is combined with the carrier in any convenient and practical manner, i.e., by solution, suspension, emulsification, admixture, encapsulation, microencapsulation, absorption and the like. Such procedures are routine for those skilled in the art.

In a specific embodiment, the fusion protein composition in powder form is combined or mixed thoroughly with a semi-solid or solid carrier. The mixing can be carried out in any convenient manner such as grinding. Stabilizing agents can be also added in the mixing process in order to protect the composition from loss of therapeutic activity through, i.e., denaturation in the stomach. Examples of stabilizers for use in an orally administrable composition include buffers, antagonists to the secretion of stomach acids, amino acids such as glycine and lysine, carbohydrates such as dextrose, mannose, galactose, fructose, lactose, sucrose, maltose, sorbitol, mannitol, etc., proteolytic enzyme inhibitors, and the like. More preferably, for an orally administered composition, the stabilizer can also include antagonists to the secretion of stomach acids.

Further, the fusion protein composition for oral administration which is combined with a semi-solid or solid carrier can be further formulated into hard or soft shell gelatin capsules, tablets, or pills. More preferably, gelatin capsules, tablets, or pills are enterically coated. Enteric coatings prevent denaturation of the composition in the stomach or upper bowel where the pH is acidic. See, i.e., U.S. Pat. No. 5,629,001. Upon reaching the small intestines, the basic pH therein dissolves the coating and permits the composition to be released to interact with intestinal cells, e.g., Peyer's patch M cells.

In another embodiment, the fusion protein composition in powder form is combined or mixed thoroughly with materials that create a nanoparticle encapsulating the fusion protein or to which the fusion protein is attached. Each nanoparticle will have a size of less than or equal to 100 microns. The nanoparticle may have mucoadhesive properties that allow for gastrointestinal absorption of a fusion protein that would otherwise not be orally bioavailable.

In another embodiment, a powdered composition is combined with a liquid carrier such as, i.e., water or a saline solution, with or without a stabilizing agent.

A specific fusion protein formulation that may be used is a solution of fusion protein in a hypotonic phosphate based buffer that is free of potassium where the composition of the buffer is as follows: 6 mM sodium phosphate monobasic monohydrate, 9 mM sodium phosphate dibasic heptahydrate, 50 mM sodium chloride, pH 7.0.+/−0.1. The concentration of fusion protein in a hypotonic buffer may range from 10 microgram/ml to 100 milligram/ml. This formulation may be administered via any route of administration, for example, but not limited to intravenous administration.

Further, a fusion protein composition for topical administration which is combined with a semi-solid carrier can be further formulated into a cream or gel ointment. A preferred carrier for the formation of a gel ointment is a gel polymer. Preferred polymers that are used to manufacture a gel composition of the present invention include, but are not limited to carbopol, carboxymethyl-cellulose, and pluronic polymers. Specifically, a powdered fusion protein is combined with an aqueous gel containing an polymerization agent such as Carbopol 980 at strengths between 0.5% and 5% wt/volume for application to the skin for treatment of disease on or beneath the skin. The term “topical administration” as used herein includes application to a dermal, epidermal, subcutaneous or mucosal surface.

Further, a fusion protein composition can be formulated into a polymer for subcutaneous or subdermal implantation. A preferred formulation for the implantable drug-infused polymer is an agent Generally Regarded as Safe and may include, for example, cross-linked dextran (Samantha Hart, Master of Science Thesis, “Elution of Antibiotics from a Novel Cross-Linked Dextran Gel: Quantification” Virginia Polytechnic Institute and State University, Jun. 8, 2009) dextran-tyramine (Jin, et al. (2010) Tissue Eng. Part A. 16(8):2429-40), dextran-polyethylene glycol (Jukes, et al. (2010) Tissue Eng. Part A., 16(2):565-73), or dextran-gluteraldehyde (Brondsted, et al. (1998) J. Controlled Release, 53:7-13). One skilled in the art will know that many similar polymers and hydrogels can be formed incorporating the fusion protein fixed within the polymer or hydrogel and controlling the pore size to the desired diameter.

Upon formulation, solutions are administered in a manner compatible with the dosage formulation and in such amount as is therapeutically effective to result in an improvement or remediation of the symptoms. The formulations are easily administered in a variety of dosage forms such as ingestible solutions, drug release capsules and the like. Some variation in dosage can occur depending on the condition of the subject being treated. The person responsible for administration can, in any event, determine the appropriate dose for the individual subject. Moreover, for human administration, preparations meet sterility, general safety and purity standards as required by FDA Center for Biologics Evaluation and Research standards.

The route of administration will vary, naturally, with the location and nature of the disease being treated, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration.

The term “parenteral administration” as used herein includes any form of administration in which the compound is absorbed into the subject without involving absorption via the intestines. Exemplary parenteral administrations that are used in the present invention include, but are not limited to intramuscular, intravenous, intraperitoneal, intratumoral, intraocular, nasal or intraarticular administration.

### Therapeutic Applications of Fusion Proteins

Based on rational design the fusion proteins of the present invention will serve as important biopharmaceuticals for treating autoimmune diseases and for modulating immune function in a variety of other contexts such as bioimmunotherapy for cancer and inflammatory diseases. Conditions included among those that may be effectively treated by the compounds that are the subject of this invention include an inflammatory disease with an imbalance in cytokine networks, an autoimmune disorder mediated by pathogenic autoantibodies or autoaggressive T cells, or an acute or chronic phase of a chronic relapsing autoimmune, inflammatory, or infectious disease or process.

The general approach to therapy using the isolated fusion proteins described herein is to administer to a subject having a disease or condition, a therapeutically effective amount of the isolated immunologically active fusion protein to effect a treatment. In some embodiments, diseases or conditions may be broadly categorized as inflammatory diseases with an imbalance in cytokine networks, an autoimmune disorder mediated by pathogenic autoantibodies or

The term “treating” and “treatment” as used herein refers to administering to a subject a therapeutically effective amount of a fusion protein of the present invention so that the subject has an improvement in a disease or condition, or a symptom of the disease or condition. The improvement is any improvement or remediation of the disease or condition, or symptom of the disease or condition. The improvement is an observable or measurable improvement, or may be an improvement in the general feeling of well-being of the subject. Thus, one of skill in the art realizes that a treatment may improve the disease condition, but may not be a complete cure for the disease. Specifically, improvements in subjects may include one or more of: decreased inflammation; decreased inflammatory laboratory markers such as C-reactive protein; decreased autoimmunity as evidenced by one or more of: improvements in autoimmune markers such as autoantibodies or in platelet count, white cell count, or red cell count, decreased rash or purpura, decrease in weakness, numbness, or tingling, increased glucose levels in patients with hyperglycemia, decreased joint pain, inflammation, swelling, or degradation, decrease in cramping and diarrhea frequency and volume, decreased angina, decreased tissue inflammation, or decrease in seizure frequency; decreases in cancer tumor burden, increased time to tumor progression, decreased cancer pain, increased survival or improvements in the quality of life; or delay of progression or improvement of osteoporosis.

The term “therapeutically effective amount” as used herein refers to an amount that results in an improvement or remediation of the symptoms of the disease or condition.

As used herein, “prophylaxis” can mean complete prevention of the symptoms of a disease, a delay in onset of the symptoms of a disease, or a lessening in the severity of subsequently developed disease symptoms.

The term “subject” as used herein, is taken to mean any mammalian subject to which fusion proteins of the present invention are administered according to the methods described herein. In a specific embodiment, the methods of the present disclosure are employed to treat a human subject. The methods of the present disclosure may also be employed to treat non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds (e.g., chickens, turkeys, and ducks), fish and reptiles to produce species-specific or chimeric fusion protein molecules.

In particular, the fusion proteins of the present invention may be used to treat conditions including but not limited to congestive heart failure (CHF), vasculitis, rosacea, acne, eczema, myocarditis and other conditions of the myocardium, systemic lupus erythematosus, diabetes, spondylopathies, synovial fibroblasts, and bone marrow stroma; bone loss; Paget's disease, osteoclastoma; multiple myeloma; breast cancer; disuse osteopenia; malnutrition, periodontal disease, Gaucher's disease, Langerhans' cell histiocytosis, spinal cord injury, acute septic arthritis, osteomalacia, Cushing's syndrome, monoostotic fibrous dysplasia, polyostotic fibrous dysplasia, periodontal reconstruction, and bone fractures; sarcoidosis; osteolytic bone cancers, lung cancer, kidney cancer and rectal cancer, bone metastasis, bone pain management, and humoral malignant hypercalcemia, ankylosing spondylitis and other spondyloarthropathies; transplantation rejection, viral infections, hematologic neoplasias and neoplastic-like conditions for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemialymphoma, thymoma, tumors of the mature T and NK cells, including peripheral T-cell leukemias, adult T-cell leukemia/T-cell lymphomas and large granular lymphocytic leukemia, Langerhans cell histiocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia, tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, tumors of the vascular system (angiosarcoma and hemangiopericytoma)) or other cancer.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, lymphangiosarcoma, lymphangioendotheliosarcoma, leiomyosarcoma, rhabdomyosarcoma, fibrosarcoma, myxosarcoma, chondrosarcoma), neuroendocrine tumors, mesothelioma, chordoma, synovioma, schwanoma, meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwanoma, and other carcinomas, as well as head and neck cancer.

The fusion proteins of the present invention may be used to treat autoimmune diseases. The term “autoimmune disease” as used herein refers to a varied group of more than 80 diseases and conditions. In all of these diseases and conditions, the underlying problem is that the body's immune system attacks the body itself. Autoimmune diseases affect all major body systems including connective tissue, nerves, muscles, the endocrine system, skin, blood, and the respiratory and gastrointestinal systems. Autoimmune diseases include, for example, systemic lupus erythematosus, rheumatoid arthritis, multiple sclerosis, myasthenia gravis, and type 1 diabetes.

The disease or condition treatable using the compositions and methods of the present invention may be a hematoimmunological process, including but not limited to Idiopathic Thrombocytopenic Purpura, alloimmune/autoimmune thrombocytopenia, Acquired immune thrombocytopenia, Autoimmune neutropenia, Autoimmune hemolytic anemia, Parvovirus B19-associated red cell aplasia, Acquired antifactor VIII autoimmunity, acquired von Willebrand disease, Multiple Myeloma and Monoclonal Gammopathy of Unknown Significance, Sepsis, Aplastic anemia, pure red cell aplasia, Diamond-Blackfan anemia, hemolytic disease of the newborn, Immune-mediated neutropenia, refractoriness to platelet transfusion, neonatal, post-transfusion purpura, hemolytic uremic syndrome, systemic Vasculitis, Thrombotic thrombocytopenic purpura, or Evan's syndrome.

The disease or condition may also be a neuroimmunological process, including but not limited to Guillain-Barre syndrome, Chronic Inflammatory Demyelinating Polyradiculoneuropathy, Paraproteinemic IgM demyelinating Polyneuropathy, Lambert-Eaton myasthenic syndrome, Myasthenia gravis, Multifocal Motor Neuropathy, Lower Motor Neuron Syndrome associated with anti-/GMI, Demyelination, Multiple Sclerosis and optic neuritis, Stiff Man Syndrome, Paraneoplastic cerebellar degeneration with anti-Yo antibodies, paraneoplastic encephalomyelitis, sensory neuropathy with anti-Hu antibodies, epilepsy, Encephalitis, Myelitis, Myelopathy especially associated with Human T-cell lymphotropic virus-1, Autoimmune Diabetic Neuropathy, Alzheimer's disease, Parkinson's disease, Huntingdon's disease, or Acute Idiopathic Dysautonomic Neuropathy.

The disease or condition may also be a Rheumatic disease process, including but not limited to Kawasaki's disease, Rheumatoid arthritis, Felty's syndrome, ANCA-positive Vasculitis, Spontaneous Polymyositis, Dermatomyositis, Antiphospholipid syndromes, Recurrent spontaneous abortions. Systemic Lupus Erythematosus, Juvenile idiopathic arthritis, Raynaud's, CREST syndrome, or Uveitis.

The disease or condition may also be a dermatoimmunological disease process, including but not limited to Toxic Epidermal Necrolysis, Gangrene, Granuloma, Autoimmune skin blistering diseases including Pemphigus vulgaris, Bullous Pemphigoid, Pemphigus foliaceus, Vitiligo, Streptococcal toxic shock syndrome, Scleroderma, systemic sclerosis including diffuse and limited cutaneous systemic sclerosis, or Atopic dermatitis (especially steroid dependent).

The disease or condition may also be a musculoskeletal immunological disease process, including but not limited to Inclusion Body Myositis, Necrotizing fasciitis. Inflammatory Myopathies, Myositis, Anti-Decorin (BJ antigen) Myopathy, Paraneoplastic Necrotic Myopathy, X-linked Vacuolated Myopathy, Penacillamine-induced Polymyositis, Atherosclerosis, Coronary Artery Disease, or Cardiomyopathy.

The disease or condition may also be a gastrointestinal immunological disease process, including but not limited to pernicious anemia, autoimmune chronic active hepatitis, primary biliary cirrhosis, Celiac disease, dermatitis herpetiformis, cryptogenic cirrhosis, Reactive arthritis, Crohn's disease, Whipple's disease, ulcerative colitis, or sclerosing cholangitis.

The disease or condition may also be Graft Versus Host Disease, Antibody-mediated rejection of the graft, Post-bone marrow transplant rejection, Post-infectious disease inflammation, Lymphoma, Leukemia, Neoplasia, Asthma, Type 1 Diabetes mellitus with anti-beta cell antibodies, Sjogren's syndrome, Mixed Connective Tissue Disease, Addison's disease, Vogt-Koyanagi-Harada Syndrome, Membranoproliferative glomerulonephritis. Goodpasture's syndrome, Graves' disease, Hashimoto's thyroiditis, Wegener's granulomatosis, micropolyarterits, Churg-Strauss syndrome, Polyarteritis nodosa or Multisystem organ failure.

The fusion proteins disclosed herein may also be readily applied to alter immune system responses in a variety of contexts to affect specific changes in immune response profiles. Altering or modulating an immune response in a subject refers to increasing, decreasing or changing the ratio or components of an immune response. For example, cytokine production or secretion levels may be increased or decreased as desired by targeting the appropriate combination of cytokine receptors with a fusion protein designed to interact with those receptors. The immune response may also be an effector function of an immune cell expressing a receptor of the multimerized protein, peptide or small molecule, including increased or decreased phagocytic potential of monocyte macrophage derived cells, increased or decreased osteoclast function, increased or decreased antigen presentation by antigen-presenting cells (e.g. DCs), increased or decreased NK cell function, increased or decreased B-cell function, as compared to an immune response which is not modulated by a fusion protein disclosed herein.

The fusion proteins described herein may be used to modulate expression of co-stimulatory molecules from an immune cell, including a dendritic cell, a macrophage, an osteoclast, a monocyte, or an NK cell or to inhibit in these same immune cells' differentiation, maturation, or of decreasing cytokine secretion, including interleukin-12 (IL-12), or interleukin-23 (IL-23) or of increasing cytokine secretion, including interleukin-10 (IL-10), or interleukin-6 (IL-6). A skilled artisan may also validate the efficacy of a fusion protein by exposing an immune cell to the fusion protein and measuring modulation of the immune cell function, wherein the immune cell is a dendritic cell, a macrophage, an osteoclast, an NK cell, or a monocyte. In one embodiment the immune cell is exposed to the fusion protein in vitro and further comprising the step of determining an amount of a cell surface receptor or of a cytokine production, wherein a change in the amount of the cell surface receptor or the cytokine production indicates a modulation of the immune cell function. In another embodiment the immune cell is exposed to the fusion protein in vivo in a model animal for an autoimmune disease further comprising a step of assessing a degree of improvement in the autoimmune disease.

### EXAMPLES

### Example 1

### Construct Design of Immunologically Active IgG2 Hinge (2Hinge)—Multimers

A. CTLA-4

The 2-hinge CTLA fusion construct is engineered using PCR. A cDNA clone containing the CTLA4 extracellular domain is obtained either from a commercial vendor of which there are many (Origen catalog #SC303605 encoding Homo sapiens cytotoxic T-lymphocyte-associated protein 4 (CTLA4), transcript variant 1 NM_{—}005214.3) or by synthesizing the cDNA at a commercial vendor of which there are many (DNA2.0 Menlo Park Calif.). Alternatively the cDNA can be obtained by PCR from a cDNA library of which there are many commercial vendors (Invitrogen cat#10425-015 SuperScript® Human Spleen cDNA Library). Primers complementary to the human IgG2 hinge sequence and additionally containing DNA sequences complementary to the CTLA cDNA domain are used by PCR to generate a fusion PCR product encoding the human IgG2 hinge domain and the sequence encoding the CTLA4 extracellular domain. The PCR fragment is then cloned into one of many commercial available expression vectors (pcDNA™ 3.3-TOPO® vector, Invitrogen). Alternatively restriction enzyme recognition sites can be added to the PCR primers to facilitate further manipulation and subcloning of the insert. A stop codon is added before the restriction site of the C terminal primer to prevent read through of flanking sequences for this construct. Generating the DNA fragment encoding the IgG2 hinge fusion by PCR allows for placing the 2-hinge either N-terminal or C-terminal to the fusion partner or by using a two-step PCR method using overlapping primers to position the 2-hinge internally in the fusion partner or between two separate fusion partners. It also allows for incorporating leader peptides facilitating the secretion of fusion proteins by incorporating DNA sequences encoding leader peptides in the 5-prime PCR primers.

The above example uses DNA sequences encoding the extracellular domain of the CTLA4. Alternatively we will use the complete CTLA4 sequence or we will use sequences encoding other proteins where the receptor binding and or biological function can be improved by multimerization. As mentioned in the previous example we can use the complete coding sequence, the sequence encoding extracellular domains or smaller sequences encoding receptor binding peptide domains allowing for generating smaller multivalent binding fusion proteins.

The 2-hinge CTLA construct is similarly made and contains the IgG₂ hinge and the extracellular domain of the CTLA4 as described above but also contained two epitope tags added to the C terminus of the construct. These epitope tags are used for identification or purification of the protein. In this second construct the two epitope tags, V5 and His tag, are present in frame prior to the stop codon but can also be present at the N-terminal. The purification tags can be one or several of the many protein tags used for purification and identification including the GST, myc, His and V5 tags.

B. PD-1

The 2-hinge PD-1 fusion construct is engineered using PCR. A cDNA clone containing the PD-1 extracellular domain is obtained either from a commercial vendor or by synthesizing the cDNA at a commercial vendor of which there are many (DNA2.0 Menlo Park Calif.). Alternatively the cDNA can be obtained by PCR from a cDNA library of which there are many commercial vendors. Primers complementary to the human IgG2 hinge sequence and additionally containing DNA sequences complementary to the PD-1 cDNA domain are used by PCR to generate a fusion PCR product encoding the human IgG2 hinge domain and the sequence encoding the PD-1 extracellular domain. The PCR fragment is then cloned into one of many commercially available expression vectors (pcDNA™ 3.3-TOPO® vector, Invitrogen). Alternatively restriction enzyme recognition sites can be added to the PCR primers to facilitate further manipulation and subcloning of the insert. A stop codon is added before the restriction site of the C terminal primer to prevent read through of flanking sequences for this construct. Generating the DNA fragment encoding the IgG2 hinge fusion by PCR allows for placing the 2-hinge either N-terminal or C-terminal to the fusion partner or by using a two step PCR method using overlapping primers to position the 2-hinge internally in the fusion partner or between two separate fusion partners. It also allows for incorporating leader peptides facilitating the secretion of fusion proteins by incorporating DNA sequences encoding leader peptides in the 5-prime PCR primers.

The above example uses DNA sequences encoding the extracellular domain of the PD-1. Alternatively we will use the complete PD-1 sequence or we will use sequences encoding other proteins where the receptor binding and or biological function can be improved by multimerization. As mentioned in the previous example we can use the complete coding sequence, the sequence encoding extracellular domains or smaller sequences encoding receptor binding peptide domains allowing for generating smaller multivalent binding fusion proteins.

The 2-hinge PD-1 construct is similarly made and contains the IgG₂ hinge and the extracellular domain of the PD-1 as described above but also contained two epitope tags added to the C terminus of the construct. These epitope tags are used for identification or purification of the protein. In this second construct the two epitope tags, V5 and His tag, are present in frame prior to the stop codon but can also be present at the N-terminal. The purification tags can be one or several of the many protein tags used for purification and identification including the GST, myc, His and V5 tags.

C. CERVIG Peptides

The 2-hinge CERVIG fusion construct is engineered using PCR. A cDNA clone containing the CERVIG is obtained by synthesizing the cDNA at a commercial vendor of which there are many (DNA2.0 Menlo Park Calif.). Alternatively the cDNA can be obtained by PCR. Primers complementary to the human IgG2 hinge sequence and additionally containing DNA sequences complementary to the CERVIG DNA domain are used by PCR to generate a fusion PCR product encoding the human IgG2 hinge domain. The PCR fragment is then cloned into one of many commercially available expression vectors (pcDNA™ 3.3-TOPO® vector, Invitrogen). Alternatively restriction enzyme recognition sites can be added to the PCR primers to facilitate further manipulation and subcloning of the insert. A stop codon is added before the restriction site of the C terminal primer to prevent read through of flanking sequences for this construct. Generating the DNA fragment encoding the IgG2 hinge fusion by PCR allows for placing the 2-hinge either N-terminal or C-terminal to the fusion partner or by using a two step PCR method using overlapping primers to position the 2-hinge internally in the fusion partner or between two separate fusion partners. It also allows for incorporating leader peptides facilitating the secretion of fusion proteins by incorporating DNA sequences encoding leader peptides in the 5-prime PCR primers. These fusion proteins produce high molecule weight multimers compared to constructs with no IgG2 hinge multimers. These multimers were visualized by non-reducing SDS-PAGE.

D. FCs Engineered to have an Antigen Binding Site

The 2-hinge Fc/Her2neu fusion construct is engineered using PCR. A cDNA clone containing the Fc domain engineered to have a Her2/neu binding site (SEQ ID NO: 18) is obtained by synthesizing the cDNA at a commercial vendor of which there are many (DNA2.0 Menlo Park Calif.). Alternatively the cDNA can be obtained by PCR. Primers complementary to the human IgG2 hinge sequence and additionally containing DNA sequences complementary to the Fc/Her2neu domain are used by PCR to generate a fusion PCR product encoding the human IgG2 hinge domain. The PCR fragment is then cloned into one of many commercially available expression vectors (pcDNA™ 3.3-TOPO® vector, Invitrogen). Alternatively restriction enzyme recognition sites can be added to the PCR primers to facilitate further manipulation and subcloning of the insert. A stop codon is added before the restriction site of the C terminal primer to prevent read through of flanking sequences for this construct. Generating the DNA fragment encoding the IgG2 hinge fusion by PCR allows for placing the 2-hinge either N-terminal or C-terminal to the fusion partner or by using a two step PCR method using overlapping primers to position the 2-hinge internally in the fusion partner or between two separate fusion partners. It also allows for incorporating leader peptides facilitating the secretion of fusion proteins by incorporating DNA sequences encoding leader peptides in the 5-prime PCR primers. These fusion proteins produce high molecule weight multimers compared to constructs with no IgG2 hinge multimers. These multimers were visualized by non-reducing SDS-PAGE.

### Example 2

### Expression of Recombinant Proteins

Numerous expression systems exist that are suitable for use in producing the compositions discussed above. Eukaryote-based systems in particular can be employed to produce nucleic acid sequences, or their cognate polypeptides, proteins and peptides. Many such systems are commercially and widely available.

In a particular embodiment, the 2-hinge multimers described herein are produced using Chinese Hamster Ovary (CHO) cells which are well established for the recombinant production of immunoglobulin proteins following standardized protocols. Alternatively, for example, transgenic animals may be utilized to produce the human 2-hinge multimers described herein, generally by expression into the milk of the animal using well established transgenic animal techniques. Lonberg N. Human antibodies from transgenic animals. Nat Biotechnol. 2005 September; 23(9):1117-25; Kipriyanov S M, Le Gall F. Generation and production of engineered antibodies. Mol Biotechnol. 2004 January; 26(1):39-60; See also Ko K, Koprowski H. Plant biopharming of monoclonal antibodies. Virus Res. 2005 July; 111(1):93-100.

The insect cell/baculovirus system can produce a high level of protein expression of a heterologous nucleic acid segment, such as described in U.S. Pat. Nos. 5,871,986, 4,879,236, both incorporated herein by reference in their entirety, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which utilizes a synthetic ecdysone-inducible receptor. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express vectors such as an expression construct described herein, to produce its encoded nucleic acid sequence or its cognate polypeptide, protein, or peptide. See, generally, Recombinant Gene Expression Protocols By Rocky S. Tuan, Humana Press (1997), ISBN 0896033333; Advanced Technologies for Biopharmaceutical Processing By Roshni L. Dutton, Jeno M. Scharer, Blackwell Publishing (2007), ISBN 0813805171; Recombinant Protein Production With Prokaryotic and Eukaryotic Cells By Otto-Wilhelm Merten, Contributor European Federation of Biotechnology, Section on Microbial Physiology Staff, Springer (2001), ISBN 0792371372.

### Example 3

### Expression and Purification of Immunologically Active 2-Hinge Fusion Proteins

Nucleic acid constructs described in Examples 1 and 2 are transfected into cell lines that do not naturally express the 2hinge recombinant chimerics. The encoded polypeptides are expressed as secreted proteins due to their secretory leader sequences, which generally are removed by endogenous proteases during transport out of the cells or may be subsequently cleaved and removed by techniques well known in the art. These secreted immunologically active biomimetics are purified using Protein A or protein G affinity chromatographic columns in case they are engineered to contain an Fc domain. Protein A and protein G purification is well known in the art and multiple commercial vendors exist. In case the IgG2 multimeric fusion protein contains alternative protein tags like His, myc or V5 tags these tags can be utilized for purification. Alternatively for non-tagged IgG2 fusion multimeric proteins purification can be achieved by traditional purification methods like ion-exchange, gel-filtration and hydrophobic interaction column chromatography. His tag chromatographic approaches and other affinity chromatographic purification methods are well known in the art. Size and purity of the purified 2-hinge fusion protein is verified by reducing and/or non-reducing SDS PAGE (sodium dodecyl sulfate polyacrylamide gel electrophoresis).

### Example 4

### Expression and Purification of Immunologically Active IgG2 Hinge Multimers for Large Scale Production

While various systems can be used to produce large amounts of a specific protein including bacteria, insect cells or yeast, expression in mammalian cells can minimize problems due to altered glycosylation of the proteins. Mammalian cells like CHO cells have been used to overproduce various proteins fused to an Ig backbone. In case the IgG2 hinge fusion protein contains a Fc domain in the construct the Fc domain becomes a tag that permits subsequent purification from the cell supernatant using protein affinity column purification (Harris, C L, D M Lublin and B P Morgan Efficient generation of monoclonal antibodies for specific protein domains using recombinant immunoglobulin fusion proteins: pitfalls and solutions, J. Immunol. Methods 268:245-258, 2002). Many fusion proteins are directly cloned in frame with the constant region of Ig, specifically the CH2 and CH3 partial Fc domain monomers. A specific example of expression of interferon gamma receptor extracellular domain being expressed with Ig has been used to produce large amounts of the protein with functional activity (Fountoulakis, M, C. Mesa, G. Schmid, R. Gentz, M. Manneberg, M. Zulauf, Z. Dembic and G. Garotta, Interferon gamma receptor extracellular domain expressed as IgG fusion protein in Chinese hamster ovary cells: Purification, biochemical, characterization and stoichiometry of binding, J. Biol. Chem. 270:3958-3964, 1995).

### Example 5

### Therapeutic Utility in Mouse Model of Arthritis

The therapeutic murine Collagen-Induced Arthritis (“CIA”) model is a well-established and predictive model for the efficacy of therapeutic compounds in rheumatoid arthritis. This model is well suited to assess compounds containing multimerized CTLA4-2hinge as a therapeutic drug (“Example 5 Test Article”). In this model, collagen is injected and thereby arthritis is induced in the mouse. Drugs can be assessed for the ability to ameliorate or reverse worsening arthritis. At day 0 DBA1/J mice, with the exception of one negative control group, will be injected with bovine Type II collagen solution in a 1:1 mixture with Complete Freund's Adjuvant. At day 20 the collagen-immunization will be repeated except for one control group of 10 animals that will receive no collagen and are expected not to develop arthritis. From day 22-27 the mice that will have received collagen injections will be scored every other day for development of arthritis. Each paw will receive a score as follows: 0=no visible effects of arthritis; 1=edema and/or erythema of 1 digit; 2=edema and/or erythema of 2 digits; 3=edema and/or erythema of more than 2 digits; 4=severe arthritis of entire paw and digits. A calculated Arthritic Index (AI) score will be obtained by addition of individual paw scores and recorded at each measurement with a maximum AI=16. Mice will be selected into groups for treatment when they have an AI score of 3 and grouped for treatment. On the day when a group with an AI=3 is formed, testing with compound will begin in that group. A second control group will receive no therapeutic treatment. In this experiment, groups will be individually treated with: a) Example 5 Test Article 10 mg/Kg administered IV every 4 days, b) Example 5 Test Article 30 mg/Kg administered IV every 4 days, c) Example 5 Test Article 50 mg/Kg administered IV every 4 days, d) Example 5 Test Article 100 mg/Kg administered IV every 4 days, and e) etanercept 10 mg/Kg administered IV every 2 days. Each group will be treated for 4 doses at treatment days 0, 4, 8, and 12 and the mice observed through day 21. The mice receiving Example 5 Test Article will have AI scores significantly lower than the no treatment control group and comparable or better in comparison with the etanercept-treated group and will demonstrate a dose-response relationship.

### Example 6

### Therapeutic Utility in Mouse Model of Arthritis

The CIA experiment of Example 5 will be repeated with compounds containing multimerized p40-IgG2 hinge as a therapeutic drug (“Example 6 Test Article”). In this experiment, groups will be individually treated with: a) Example 6 Test Article 10 mg/Kg administered IV every 4 days, b) Example 6 Test Article 30 mg/Kg administered IV every 4 days, c) Example 6 Test Article 50 mg/Kg administered IV every 4 days, d) Example 6 Test Article 100 mg/Kg administered IV every 4 days, and e) prednisolone 10 mg administered orally daily. Each Test Article group will be treated for 4 doses at treatment days 0, 4, 8, and 12 and the mice observed through day 21. The mice receiving Example 6 Test Article will have AI scores significantly lower than the no treatment control group and comparable or better in comparison with the steroid-treated group and will demonstrate a dose-response relationship.

### Example 7

### Diagnostic Utility in Flow Cytometry

Flow cytometry is a technique for counting and examining microscopic particles, such as cells, by suspending them in a stream of fluid and passing them by an electronic detector. It allows simultaneous multiparametric analysis of the physical and/or chemical characteristics of up to thousands of particles per second. Flow cytometry is routinely used in the diagnosis of health disorders but has many other applications in both research and clinical practice. A common research use is to physically sort particles based on their properties, such as a cell surface marker, so as to purify populations of interest.

As MHC tetramer, which is frequently created via the use of biotin-streptavidin affinity among the MHC units, is a useful reagent in performing flow cytometry, especially for T-cells, so multimerized MHC fused to the IgG2 hinge will be of even greater utility. A peptide from the antigen of interest will be attached to the MHC-IgG2 hinge fusion protein and the protein will be fluorescently labeled. Just as each MHC tetramer must be custom-made for each antigen-specific T-cell that one desires to detect, so each IgG2 hinge-MHC multimer must similarly be custom made. The fluorescent MHC-antigen-IgG2 hinge multimer will bind only the specific T-cells that respond to that peptide. The multimerized IgG2 hinge-MHC-antigen will be able to be detected by flow cytometry via the fluorescent label at sensitivities as great as, or greater than, a tetramer comprised of the same MHC and combined by biotin-Streptavidin binding.

1. A fusion protein comprising:
one or more IgG2 hinge monomers; and
one or more peptides, proteins, carbohydrates/sugars or small molecules
wherein said one or more IgG2 hinge domains multimerizes said one or more peptides, proteins or small molecules into dimers or higher order multimers.

2. The fusion protein of claim 1, wherein said one or more peptides, proteins or small molecules is any peptide, protein, small molecule or carbohydrate/sugar whose activity is improved by multimerization.

3. The fusion protein of claim 2, wherein said protein or peptide is selected from the group consisting of cytokines, chemokines, hormones, monoclonal antibodies and antibody-like compounds, cell surface receptors, cell surface receptor ligands and fragments thereof.

4. The fusion protein of claim 2 wherein the protein is selected from the group consisting of PD-1, PDL-1L, CERVIG peptide, CTLA4, IL12, IL12RA, major histocompatibility complex and insulin.

5. The fusion protein of claim 2, wherein said peptide is selected from the group consisting of the external domain of PD-1, CTLA4, the p40 subunit of IL12, and human parathyroid hormone

6. The fusion protein of claim 2, wherein said small molecule is selected from the group consisting of chemotherapeutic agents, cytotoxic molecules, dyes and flurophores.

7. The fusion protein of claim 2, wherein the carbohydrate/sugar is selected from the group consisting of monosaccharides, disaccharides, oligosaccharides, polysaccharides, neoglycoproteins, glycoclusters, glycopolymers, monodisperse nanostructures termed glycodendrimers, sugar alcohols, and sugar-rods.

8. The fusion protein of claim 1, wherein the IgG2 hinge is at least 80% homologous to SEQ ID NO:1.

9. The fusion protein of claim 1, wherein the IgG2 hinge is at least 90% homologous to SEQ ID NO:1.

10. The fusion protein of claim 1, wherein the IgG2 hinge is at least 95% homologous to SEQ ID NO:1.

11. The fusion protein of claim 1, wherein the IgG2 hinge is 100% homologous to SEQ ID NO: 1.

12. The fusion protein of claim 1, wherein the IgG2 hinge comprises at least one C-X-X-C motifs.

13. The fusion protein of claim 1, wherein the IgG2 hinge comprises at least two C-X-X-C motifs.

14. The fusion protein of claim 12 or 13, wherein the X-X in the said C-X-X-C motif comprises V-E or P-P.

15. The fusion protein of claim 1, further comprising an immunoglobulin Fc domain.

16. The fusion protein of claim 15, wherein said immunoglobulin Fc domain is selected for poor binding to Fc gamma receptors.

17. The fusion protein of claim 16, wherein the Fc domain is an IgG1, IgG2, IgG3 or IgG4 Fc domain.

18. The fusion protein of claim 15, wherein said immunoglobulin Fc domain is mutated to bind poorly to Fc gamma receptors.

19. The fusion protein of claim 18, wherein said Fc domain is mutated at one or more of positions 233, 234, 235, 236, 238, 239, 265, 269, 270, 292, 293, 295, 296, 297, 303, 327, 329, 338, 376, and/or 414.

20. The fusion protein of claim 15, wherein said Fc domain is modified to bind poorly to an Fc gamma receptor.

21. The fusion protein of claim 20, wherein said Fc domain is modified by hyperfucosylation, demannosylation or hemi-glycosylation.

22. A pharmaceutical formulation comprising the fusion protein of claim 1, and pharmaceutically acceptable excipients.

23. A method of treating a patient in need thereof with the pharmaceutical formulation of claim 22.

24. The method of claim 23, wherein said patient has an inflammatory disease.

25. The method of claim 24, wherein the inflammatory disease is an autoimmune disease.

26. The method of claim 25, wherein the autoimmune disease is arthritis, multiple sclerosis, type I diabetes, autoimmune thyroiditis, idiopathic thrombocytopenic purpura, chronic inflammatory polyneuropathy, multifocal motor neuropathy, scleroderma, autoimmune uveitis, systemic lupus erythmatosis, myasthenia gravis, rheumatoid arthritis, Crohn's disease, and atopic dermatitis.

27. The method of claim 25, wherein the autoimmune disease is associated with the transplantation of an organ from a donor to a recipient.

28. The method of claim 24, wherein the inflammatory disease is an infectious disease.

29. The method of claim 28, wherein the infectious disease is a bacterial or fungal infection.

30. The method of claim 28, wherein the infectious disease is a viral infection.

31. The method of claim 23, wherein the fusion protein is administered to the patient intravenously, subcutaneously, orally, intraperitoneally, sublingually, bucally, transdermally, by subdermal implant, or intramuscularly.

32. The method of claim 31, wherein the fusion protein is administered intravenously.

33. The method of claim 23, further comprising administering an additional pharmaceutically active agent.

34. The method of claim 33, wherein the additional pharmaceutically active agent comprises a steroid, a monoclonal antibody, an antibiotic, an anti-viral agent, a cytokine, or an agent otherwise capable of acting as an immune modulator.

35. The method of claim 35, wherein the steroid is prednisolone, cortisone, mometesone, testosterone, estrogen, oxandrolone, fluticasone, budesonide, beclamethasone, albuterol, or levalbuterol.

36. A fusion protein comprising:
one or more IgG2 hinge monomers; and
one or more Fc domains, wherein said one or more Fc domains has been engineered to comprise an antigen binding site;
wherein said one or more IgG2 hinge domains multimerizes said one or more peptides, proteins or small molecules into dimers or higher order multimers.

37. The fusion protein of claim 36 wherein said fusion protein is capable of binding:
a) FcγR, complement, or FcRn; and
b) the antigen for which the Fc domain has been engineered to bind.

38. The fusion protein of claim 36 wherein the antigen for which the Fc domain has been engineered to bind is Her2/neu.

39. The fusion protein of claim 36, wherein the IgG2 hinge is at least 80% homologous to SEQ ID NO:1.

40. The fusion protein of claim 36, wherein the IgG2 hinge is at least 90% homologous to SEQ ID NO:1.

41. The fusion protein of claim 36, wherein the IgG2 hinge is at least 95% homologous to SEQ ID NO:1.

42. The fusion protein of claim 36, wherein the IgG2 hinge is 100% homologous to SEQ ID NO: 1.

43. The fusion protein of claim 36, wherein the IgG2 hinge comprises at least one C-X-X-C motifs.

44. The fusion protein of claim 36, wherein the IgG2 hinge comprises at least two C-X-X-C motifs.

45. The fusion protein of claim 43 or 44, wherein the X-X in the said C-X-X-C motif comprises V-E or P-P.

46. A pharmaceutical formulation comprising the fusion protein of claim 36, and pharmaceutically acceptable excipients.

47. A method of treating a patient in need thereof with the pharmaceutical formulation of claim 22.

48. The method of claim 47, wherein said patient has an inflammatory disease.

49. The method of claim 48, wherein the inflammatory disease is an autoimmune disease.

50. The method of claim 49, wherein the autoimmune disease is arthritis, multiple sclerosis, type I diabetes, autoimmune thyroiditis, idiopathic thrombocytopenic purpura, chronic inflammatory polyneuropathy, scleroderma, autoimmune uveitis, systemic lupus erythmatosis, myasthenia gravis, and atopic dermatitis.

51. The method of claim 49, wherein the autoimmune disease is associated with the transplantation of an organ from a donor to a recipient.

52. The method of claim 48, wherein the inflammatory disease is an infectious disease.

53. The method of claim 52, wherein the infectious disease is a bacterial infection or a viral infection.

54. The method of claim 52, wherein said patient has cancer.

55. The method of claim 47, wherein the fusion protein is administered to the patient intravenously, subcutaneously, orally, intraperitoneally, sublingually, bucally, transdermally, by subdermal implant, or intramuscularly.

56. The method of claim 55, wherein the fusion protein is administered intravenously.

57. The method of claim 47, further comprising administering an additional pharmaceutically active agent.

58. The method of claim 57, wherein the additional pharmaceutically active agent comprises a steroid, a monoclonal antibody, an antibiotic, an anti-viral agent, a cytokine, or an agent otherwise capable of acting as an immune modulator.

59. The method of claim 58, wherein the steroid is prednisolone, cortisone, mometesone, testosterone, estrogen, oxandrolone, fluticasone, budesonide, beclamethasone, albuterol, or levalbuterol.

60. A fusion protein comprising:
one or more IgG2 hinge monomers; and
one or more antigen binding antibody variable domains, fragments or variants thereof;
wherein said one or more IgG2 hinge domains multimerizes said one or more antigen binding antibody variable domains, fragments or variants thereof into dimers or higher order multimers.

61. The fusion protein of claim 60 comprising a variable heavy chain (V_{H}) linked to a variable light chain (V_{L}).

62. The fusion protein of claim 61 wherein the fusion of the V_{H} to the V_{L} forms an epitope binding site.

63. The fusion protein of claim 61 wherein the V_{H} is linked to the V_{L} with a linker.

64. The fusion protein of claim 63 further comprising the CH1 region of V_{H} or V_{L}.

65. The fusion protein of claim 1 wherein the V_{H} and V_{L} are co-expressed in the same cell.

66. The fusion protein of claim 60, wherein the one or more antigen binding antibody variable domains, fragments or variants thereof is a Fab fragment, scFv, a diabody, a triabody, a minibody, a single-domain antibody, a nanobody or a single chain antibody.

67. The fusion protein of claim 60, wherein the IgG2 hinge is at least 80% homologous to SEQ ID NO:1.

68. The fusion protein of claim 60, wherein the IgG2 hinge is at least 90% homologous to SEQ ID NO:1.

69. The fusion protein of claim 60, wherein the IgG2 hinge is at least 95% homologous to SEQ ID NO:1.

70. The fusion protein of claim 60, wherein the IgG2 hinge is 100% homologous to SEQ ID NO: 1.

71. The fusion protein of claim 60, wherein the IgG2 hinge comprises at least one C-X-X-C motifs.

72. The fusion protein of claim 60, wherein the IgG2 hinge comprises at least two C-X-X-C motifs.

73. The fusion protein of claim 71 or 72, wherein the X-X in the said C-X-X-C motif comprises V-E or P-P.

74. The fusion protein of claim 1, further comprising an immunoglobulin Fc domain.

75. The fusion protein of claim 74, wherein said immunoglobulin Fc domain is selected for poor binding to Fc gamma receptors.

76. The fusion protein of claim 75, wherein the Fc domain is an IgG1, IgG2, IgG3 or IgG4 Fc domain.

77. The fusion protein of claim 74, wherein said immunoglobulin Fc domain is mutated to bind poorly to Fc gamma receptors.

78. The fusion protein of claim 77, wherein said Fc domain is mutated at one or more of positions 233, 234, 235, 236, 238, 239, 265, 269, 270, 292, 293, 295, 296, 297, 303, 327, 329, 338, 376, and/or 414.

79. The fusion protein of claim 74, wherein said Fc domain is modified to bind poorly to an Fc gamma receptor.

80. The fusion protein of claim 79, wherein said Fc domain is modified by hyperfucosylation, demannosylation or hemi-glycosylation.

81. A pharmaceutical formulation comprising the fusion protein of claim 60, and pharmaceutically acceptable excipients.

82. A method of treating a patient in need thereof with the pharmaceutical formulation of claim 81.

83. The method of claim 82, wherein said patient has an inflammatory disease.

84. The method of claim 83, wherein the inflammatory disease is an autoimmune disease.

85. The method of claim 84, wherein the autoimmune disease is arthritis, multiple sclerosis, type I diabetes, autoimmune thyroiditis, idiopathic thrombocytopenic purpura, chronic inflammatory polyneuropathy, scleroderma, autoimmune uveitis, systemic lupus erythmatosis, myasthenia gravis, and atopic dermatitis.

86. The method of claim 84, wherein the autoimmune disease is associated with the transplantation of an organ from a donor to a recipient.

87. The method of claim 83, wherein the inflammatory disease is an infectious disease.

88. The method of claim 87, wherein the infectious disease is a bacterial infection.

89. The method of claim 87, wherein the infectious disease is a viral infection.

90. The method of claim 82, wherein the fusion protein is administered to the patient intravenously, subcutaneously, orally, intraperitoneally, sublingually, bucally, transdermally, by subdermal implant, or intramuscularly.

91. The method of claim 31, wherein the fusion protein is administered intravenously.

92. The method of claim 23, further comprising administering an additional pharmaceutically active agent.

93. The method of claim 33, wherein the additional pharmaceutically active agent comprises a steroid, a monoclonal antibody, an antibiotic, an anti-viral agent, a cytokine, or an agent otherwise capable of acting as an immune modulator.

94. The method of claim 35, wherein the steroid is prednisolone, cortisone, mometesone, testosterone, estrogen, oxandrolone, fluticasone, budesonide, beclamethasone, albuterol, or levalbuterol.

95. The fusion protein of claim 1, wherein the one or more peptides, proteins, carbohydrates/sugars or small molecule is fused to the N terminus of the IgG2 hinge.

96. The fusion protein of claim 1, wherein the one or more peptides, proteins, carbohydrates/sugars or small molecule is fused to the C terminus of the IgG2 hinge.

97. The fusion protein of claim 95 wherein the one or more peptides, proteins, carbohydrates/sugars or small molecules is CTLA-4 or an extracellular domain thereof.

98. The fusion protein of claim 97, further comprising a B7.1 or B7.2 protein.

98. The fusion protein of claim 95 wherein the one or more peptides, proteins, carbohydrates/sugars or small molecules is PD-1.

99. The fusion protein of claim 98 wherein the PD-1 is the extracellular domain of PD-1.

100. The fusion protein of claim 99 comprising SEQ ID NO: 6.

101. The fusion protein of claim 95 wherein the one or more peptides, proteins, carbohydrates/sugars or small molecules is a CERVIG peptide.

102. The fusion protein of claim 101 wherein the CERVIG comprises SEQ ID NO: 11.

103. The fusion protein of claim 102 comprising SEQ ID NO: 16.

104. The fusion protein of claim 96 wherein the one or more peptides, proteins, carbohydrates/sugars or small molecules is CTLA-4 or an extracellular domain thereof.

105. The fusion protein of claim 104, further comprising a B7.1 or B7.2 protein.

106. The fusion protein of claim 96 wherein the one or more peptides, proteins, carbohydrates/sugars or small molecules is PD-1.

107. The fusion protein of claim 106 wherein the PD-1 is the extracellular domain of PD-1.

108. The fusion protein of claim 107 comprising SEQ ID NO: 5.

109. The fusion protein of claim 96 wherein the one or more peptides, proteins, carbohydrates/sugars or small molecules is a CERVIG peptide.

110. The fusion protein of claim 109 wherein the CERVIG comprises SEQ ID NO: 11.

111. The fusion protein of claim 36 wherein the IgG2 hinge is fused to the C terminus of the one or more Fc domains engineered to comprise an antigen binding site.

112. The fusion protein of claim 36 wherein the IgG2 hinge is fused to the N terminus of the one or more Fc domains engineered to comprise an antigen binding site.

113. The fusion protein of claim 111 wherein the antigen to which the Fc domain is engineered to bind is Her2/neu of SEQ ID NO: 18.

114. The fusion protein of claim 113 comprising SEQ ID NO: 19

115. The fusion protein of claim 112 wherein the antigen to which the Fc domain is engineered to bind is Her2/neu of SEQ ID NO: 18.

116. The fusion protein of claim 115 comprising SEQ ID NO: 20.

117. The fusion protein of claim 60 wherein the IgG2 hinge is fused to the C terminus of the one or more antigen binding antibody variable domains, fragments or variants thereof.

118. The fusion protein of claim 60 wherein the IgG2 hinge is fused to the N terminus of the one or more antigen binding antibody variable domains, fragments or variants thereof.

119. The fusion protein of claim 15 wherein the one or more peptides, proteins, carbohydrates/sugars or small molecules is PD-1.

120. The fusion protein of claim 119 wherein the PD-1 is the extracellular domain of PD-1.

121. The fusion protein of claim 120 comprising SEQ ID NO: 7, SEQ ID NO: 8, SEQ ID NO:9, or SEQ ID NO: 10.

122. The fusion protein of claim 15 wherein the one or more peptides, proteins, carbohydrates/sugars or small molecules is a CERVIG peptide.

123. The fusion protein of claim 122 wherein the CERVIG peptide comprises SEQ ID NO: 11.

124. The fusion protein of claim 123 comprising SEQ ID NO: 13, SEQ ID NO: 14 or SEQ ID NO: 15.

125. The fusion protein of claim 1 further comprising a linker between the one or more peptides, proteins, carbohydrates/sugars or small molecules and one or more IgG2 hinge monomers.

126. The fusion protein of claim 125 wherein the one or more peptides, proteins, carbohydrates/sugars or small molecules is a CERVIG peptide.

127. The fusion protein of claim 126 wherein the CERVIG peptide comprises SEQ ID NO: 11.

128. The fusion protein of claim 127 comprising SEQ ID NO: 17.

129. The fusion protein of claim 36 further comprising one or more additional Fc domains.

130. The fusion protein of claim 129 wherein the Fc domain that has been engineered to comprise an antigen binding site has been engineered to bind Her2/neu.

131. The fusion protein of claim 130 wherein the Fc domain that has been engineered to bind Her2/neu comprises SEQ ID NO: 18.

132. The fusion protein of claim 131 comprising SEQ ID NO: 21.

133. The fusion protein of claim 4 wherein the protein is a major histocompatibility complex.

134. The fusion protein of claim 133 wherein the major histocompatibility complex is a class I major histocompatibility complex.

135. The fusion protein of claim 133 wherein the major histocompatibility complex is a class II major histocompatibility complex.

136. The fusion protein of claim 133 further comprising a dye or a fluorophore.

137. The use of the fusion protein of claim 136 in flow cytometry to detect antigen specific T cells.

138. The fusion protein of claim 3 wherein the protein or peptide is a cell surface receptor.

139. The fusion protein of claim 138 wherein the cell surface receptor is a G-protein coupled receptor.

140. The fusion protein of claim 139 wherein the G-protein coupled receptor is a chemokine receptor.

141. The fusion protein of claim 140 wherein the chemokine receptor is CCR5, CXCR1 or CXCR2.

142. The fusion protein of claim 138 wherein the cell surface receptor is a B cell receptor.

143. The fusion protein of claim 138 wherein the cell surface receptor is a T cell receptor.

144. The fusion protein of claim 138 wherein the cell surface receptor is a TNF superfamily receptor.

145. The fusion protein of claim 144 wherein the TNF superfamily receptor is CD137, BAFF R, BCMA, CD27, CD30, CD40, DcR3, DcTRAIL, DR3, DR6, EDAR, Fas, GITR, HVEM, lyphotoxin beta R, NGF R, osteoprotegerin, OX40, RANK, RELT, TACI, TRAIL R, TROY, or TWEAK R.

146. The fusion protein of claim 145 wherein the TRAIL R is TRAIL R1, TRAIL R2, TRAIL R3 or TRAIL R4.

147. The fusion protein of claim 3 wherein the protein or peptide is a cell surface rector ligand.

148. The fusion protein of claim 147 wherein the cell surface receptor ligand is a ligand to a TNF superfamily receptor.

149. The fusion protein of claim 148 wherein the ligand to a TNF super family receptor is TNFα or BLyS.

150. The fusion protein of claim 147 wherein the cell surface receptor ligand is a ligand to a cell surface glycoprotein.

151. The fusion protein of claim 150 wherein the ligand binding to a cell surface glycoprotein is a CD4, CD123, CD303, or A CD304 ligand.
